(19) United States
(12) Patent Application Publication
(10) Pub. No.: US 2022/0265787 A1
(43) Pub. Date: Aug. 25, 2022

(54) COMPOSITION FOR PROMOTING GROWTH OR SUPPRESSING DECREASE OF MESENCHYMAL STEM CELLS

(71) Applicants: STEMRIM INC., Osaka (JP); OSAKA UNIVERSITY, Osaka (JP)

(72) Inventors: Katsuto TAMAI, Suita-shi, Osaka (JP); Takashi SHIMBO, Suita-shi, Osaka (JP); Takehiko YAMAZAKI, Ibaraki-shi, Osaka (JP)

(73) Assignees: STEMRIM INC., Osaka (JP); OSAKA UNIVERSITY, Osaka (JP)

(21) Appl. No.: 17/631,072

(22) PCT Filed: Jul. 30, 2020
(86) PCT No.: PCT/JP2020/029229

§ 371 Date: Apr. 29, 2022

(30) Foreign Application Priority Data
Jul. 31, 2019 (JP) 2019-141325
Mar. 27, 2020 (JP) 2020-058544

(51) Int. Cl. A61K 38/57; A61K 9/00; A61P 1/04; A61P 43/00; C12N 5/0775

(52) U.S. Cl. CPC A61K 38/57; A61K 9/0019; C12N 5/0668; A61P 43/00; A61P 1/04

(57) ABSTRACT

The present disclosure includes a composition for promoting growth or suppressing decrease of mesenchymal stem cells comprising serpin A3.

### TECHNICAL FIELD

The present application claims priority with respect to the Japanese Patent Applications Nos. 2019-141325 and 2020-058544, each of which is herein incorporated by reference in its entirety.

The present disclosure relates to a composition for promoting growth or suppressing decrease of mesenchymal stem cells.

### BACKGROUND

It has been reported that HMGB1 (high mobility group box 1 protein) released from damaged tissues stimulates PDGFRα (platelet-derived growth factor receptor alpha)-positive cells (mesenchymal stem cells) present in the bone marrow to mobilize the cells from the bone marrow to the circulatory system, and the mobilized cells accumulate in damaged sites to induce tissue regeneration (Patent Documents 1 and 2). Thus, if mesenchymal stem cells in the bone marrow are reduced for some reason, tissue regeneration may be delayed or inadequate. Then, in order for the tissue regeneration mechanism in the living body via mesenchymal stem cells to function effectively, it is important that the required amount of mesenchymal stem cells is present in the bone marrow. Therefore, it is desired to maintain, recover, or increase the amount of cells including mesenchymal stem cells in the bone marrow, and development of new drugs having such effects has been awaited.

Also, in recent years, the number of patients with inflammatory bowel disease has been increasing. Inflammatory bowel disease is a general term for inflammatory diseases of the intestinal tract that are chronic or repeat remission and relapse, and generally refers to two diseases, ulcerative colitis and Crohn's disease. Ulcerative colitis and Crohn's disease are both intractable diseases of unknown causes. Although they have been treated by drug therapies, there are individual differences in drug efficacy among patients. Also, for some cases, existing drugs with high therapeutic effects cannot be prescribed due to their side effects. Therefore, provision of new drugs has been awaited.

### PRIOR ART DOCUMENTS

### Patent Documents

Patent Document 1: WO/2008/053892
Patent Document 2: WO/2009/133939

### SUMMARY

### Problems to be Solved by Invention

One of objects of the present disclosure is to promote growth or suppress decrease of mesenchymal stem cells.

Another one of objects of the present disclosure is to provide a composition for treating inflammatory bowel disease.

### Means to Solve Problems

In an aspect, the present disclosure relates to a composition for promoting growth or suppressing decrease of mesenchymal stem cells comprising serpin A3.

In a further aspect, the present disclosure relates to a composition for treating inflammatory bowel disease comprising serpin A3.

### Effect of Invention

According to the present disclosure, it is possible, for example, to promote growth or suppress decrease of mesenchymal stem cells, particularly bone marrow mesenchymal stem cells. Also, according to the present disclosure, a therapeutic effect on inflammatory bowel disease can be expected, for example.

### BRIEF DESCRIPTION OF DRAWINGS

FIG. 1 shows the body weight change of healthy mice and DSS-administered mice. Each plot shows the mean value and the error bar shows the standard error. *p<0.05, **p<0.01, two-way ANOVA.

FIG. 2 shows the measurement results of colon length of healthy mice and DSS-administered mice. The left photo shows colons taken from the mice. The right graph shows the colon length measured on each collection day, and the mean value of three animals is shown by the bar in the graph and the standard error is shown by the error bar. *p<0.05, **p<0.01, two-way ANOVA.

FIG. 3 shows the colony assay results that demonstrate changes in colony-forming cells in the bone marrow of DSS-administered mice. Shown is the colony number (%) of DSS-administered mice on each collection day when the mean value of numbers of colonies formed from bone marrow cells of healthy mice on the same collection day is set as 100%. *p<0.05, **p<0.01, two-way ANOVA.

FIG. 4 shows the colony assay results when bone marrow cells of DSS-administered mice were cultured in a serpin A3N-added medium. The left graph shows the colony number when bone marrow cells of healthy mice were cultured for 10 days in the presence of serpin A3N (0, 0.5, 1, 2, or 4 ng/mL). The right graph shows the colony number when bone marrow cells of DSS-administered mice were cultured in the presence or absence of serpin A3N (4 ng/mL) for 10 days (“IBD+Seripina3n” or “IBD” in the graph), and the colony number when bone marrow cells of healthy mice were cultured for the same period in the absence of serpin A3N (“Ctrl” in the graph). *p<0.05, **p<0.01, two-way ANOVA.

FIG. 5 shows the colony assay results of bone marrow cells collected from mice treated with DSS and PBS containing no serpin A3N (“DSS+PBS” in the graph), mice treated with DSS and PBS containing serpin A3N (“DSS+Seripina3n” in the graph), or healthy mice (“Control” in the graph). *p<0.05, one-way ANOVA.

FIG. 6 shows the body weight change (top), colon photograph (bottom left), and measured colon length (bottom right) of mice treated with DSS and PBS containing no serpin A3N (“DSS+PBS” in the graph), mice treated with DSS and PBS containing serpin A3N (“DSS+Seripina3n” in the graph), or healthy mice (“Control” in the graph). Each plot in the graph of body weight change and each bar in the graph of colon length show the mean value, and the error bar shows the standard error. Arrowheads indicate administration of serpin A3N.

FIG. 7 shows the body weight change of mice treated with DSS and PBS containing no serpin A3N (“DSS+PBS” in the graph), mice treated with DSS and PBS containing serpin A3N (“DSS+Seripina3n” in the graph), or healthy mice (“Control” in the graph). Each plot shows the mean value and the error bar shows the standard error. Arrowheads indicate administration of serpin A3N.

FIG. 8 shows the body weight change of mice to which PBS containing no serpin A3N or PBS containing serpin A3N was administered after DSS administration (“DSS+PBS” or “DSS+Seripina3n” in the graph) or healthy mice (“Control” in the graph). Each plot shows the mean value and the error bar shows the standard error. Arrowheads indicate administration of serpin A3N.

FIG. 9 shows the body weight change of mice treated with DSS and PBS containing no serpin A3N (black column) or mice treated with DSS and PBS containing serpin A3N (slash column) after additional DSS administration. The result of healthy mice (white column) is shown as a control. The body weight of the mice on the 21st day from the first start date of DSS administration (the start date of additional DSS administration) is shown as 100%. The bar in the graph shows the mean value, and the error bar shows the standard error.

FIG. 10 shows the gene expression pattern in colon cells of DSS-administered mice. The left graph shows four subclusters (K1, K2, K3, K4) clustered by the K-means clustering method, and the right graph shows the expression level of each gene contained in K4.

FIG. 11 shows the expression of TNF-α, IL-1β, and IL-6 in colon cells of mice treated with DSS and PBS containing no serpin A3N (“DSS+PBS” in the graph), mice treated with DSS and PBS containing serpin A3N (“DSS+Seripina3n” in the graph), or healthy mice (“Control” in the graph). *p<0.05, one-way ANOVA.

### DETAILED DESCRIPTION OF EMBODIMENTS

Unless otherwise specified, the terms used in this disclosure have the meanings generally understood by those skilled in the art in the fields such as organic chemistry, medical science, pharmaceutical science, molecular biology, and microbiology. The followings are definitions of some of the terms used in this disclosure, and these definitions supersede the general understandings in this disclosure.

In the present disclosure, when a number is accompanied by the term “about”, it is intended to include a range of ±10% of that value. For example, “about 20” shall include “18 to 22”. The range of numbers includes all numbers between the endpoints and the numbers at the endpoints. The “about” for a range applies to both ends of the range. Therefore, for example, “about 20 to 30” shall include “18 to 33”.

In the present disclosure, the term “cell” can mean either a single cell or a plurality of cells depending on the context. A plurality of cells may be referred to as a “cell population”. The cell population may be a cell population of one type of cell or a cell population containing multiple types of cells depending on the context.

Serpin A3 or a composition comprising the same in the present disclosure promotes growth or suppresses decrease of mesenchymal stem cells in vivo or in vitro. In an embodiment, serpin A3 or a composition comprising the same is administered to a subject to promote growth or suppress decrease of mesenchymal stem cells in the subject's body. The promoting growth of cells includes increasing the number of cells in a subject, and the suppressing decrease of cells includes preventing decrease in the number of cells in a subject and reducing such decrease. In another embodiment, serpin A3 or a composition comprising the same is used to promote growth or suppress decrease of mesenchymal stem cells during in vitro culture.

The subject may be, but not limited to, a human or a non-human animal such as mouse, rat, monkey, pig, dog, rabbit, hamster, or guinea pig. In an embodiment, the subject is a human.

In the present disclosure, “mesenchymal stem cell” (also herein referred to as MSC) means a cell capable of differentiating into a mesenchymal tissue such as bone, cartilage, fat, or muscle. The mesenchymal stem cell may also have the ability to differentiate into an epithelial or neural tissue. Mesenchymal stem cells are present in the bone marrow, blood such as peripheral or umbilical cord blood, skin, fat, or pulp. In an embodiment, the mesenchymal stem cells are bone marrow mesenchymal stem cells.

In an embodiment, mesenchymal stem cells can be identified on the basis of their colony-forming ability. Mesenchymal stem cells can be reliably identified by observing the shape and size of colonies and the density and morphology of colony-forming cells.

Markers for human mesenchymal stem cells can be, but not limited to, all or part of PDGFRα positive, PDGFRβ positive, Lin negative, CD45 negative, CD44 positive, CD90 positive, CD29 positive, Flk-1 negative, CD105 positive, CD73 positive, CD90 positive, CD71 positive, Stro-1 positive, CD106 positive, CD166 positive, CD31 negative, CD271 positive, and CD11b negative. For example, human mesenchymal stem cells may be identified as PDGFRα positive, or may be identified as PDGFRα positive and CD45 negative. In an embodiment, human mesenchymal stem cells may be identified as PDGFRβ positive, or may be identified as PDGFRβ positive and CD45 negative.

Markers for mouse mesenchymal stem cells can be, but not limited to, all or part of CD44 positive, PDGFRα positive, PDGFRβ positive, CD45 negative, Lin negative, Sca-1 positive, c-kit negative, CD90 positive, CD105 positive, CD29 positive, Flk-1 negative, CD271 positive and CD11b negative. For example, mouse mesenchymal stem cells may be identified as PDGFRα positive, or may be identified as PDGFRα positive and CD45 negative.

In the present disclosure, the term “bone marrow cells” means a cell population present in the bone marrow. Bone marrow cells include cells expressing CD45 (also herein referred to as CD45-positive cells or CD45⁺ cells) and cells not expressing CD45 (also herein referred to as CD45-negative cells or CD45⁻ cells).

In the present disclosure, the bone marrow can be, but not limited to, bone marrow of the femur, tibia, skull, sternum, vertebra, costa, or pelvic bone.

In an embodiment, serpin A3 or a composition comprising the same promotes growth or suppresses decrease of colony-forming mesenchymal stem cells. The term “colony-forming mesenchymal stem cells” means mesenchymal stem cells that adhere to a solid phase to form colonies when cultured on the solid phase. Growth or decrease of colony-forming mesenchymal stem cells can be evaluated by a colony assay, for example.

In an embodiment, serpin A3 or a composition comprising the same promotes growth or suppresses decrease of bone marrow mesenchymal stem cells (i.e., mesenchymal stem cells contained in bone marrow cells). In a further embodiment, serpin A3 or a composition comprising the same promotes growth or suppresses decrease of bone marrow colony-forming mesenchymal stem cells.

Serpins are a superfamily of proteins with similar structures that were first identified for their serine protease inhibition activity and are found in all kingdoms of life. Serpins generally have the function of controlling protein degradation reaction. Regarding the mechanism of action, they have been known to irreversibly inhibit their target protease by undergoing a large conformational change to disrupt the target's active site.

Human serpin A3 is one of the serpins, also called al-antichymotrypsin, and it is a protein encoded by the human SERPINA3 gene. Serpin A3 is known to inhibit proteases such as chymotrypsin, chymase, elastase, and cathepsin G. The SERPINA3 gene is known to be conserved in chimpanzees, rhesus monkeys, dogs, bovines, mice, and rats.

In mice, some genes are known as homologs of the human SERPINA3 gene. Among these genes, the SERPINA3N gene has a high degree of homology with the human SERPINA3 gene. Serpin A3N has been reported to share the substrate specificity with each of serpin A3 and serpin A1 (also called antitrypsin) and inhibit chymotrypsin, trypsin, elastase, and cathepsin G. Serpin A3N has also been reported to inhibit granzyme B.

In the present disclosure, the term “serpin A3” means a protein encoded by the human SERPINA3 gene or a homolog thereof (herein referred to as a serpin A3 gene collectively). Serpin A3 can be, but not limited to, a human or non-human animal protein, for example a human, mouse, rat, hamster, guinea pig, rabbit, dog, pig, bovine, monkey, chimpanzee, or orangutan protein. Examples of proteins encoded by homologs of the human SERPINA3 gene include, but are not limited to, proteins encoded by the genes shown in Table 1.

TABLE 1-1

| | | NCBI Reference | | | | | |
|---|---|---|---|---|---|---|---|
| | | Sequence | Entrez | Amino | | | |
| | Gene | (VERSION) | gene ID | acids | signal | region | region name |
| human | SERPINA3 | NP_00076.2 | 12 | 423 | 1..23 | 52..417 | alpha-1- |
| | | NM_001085.5 | | | | | antitrypsin_like |
| mouse | Serpina3a | NP_001161177.1 | 74069 | 422 | 1..17 | 53..416 | SERPIN |
| | Serpina3b | NP_766612.1 | 271047 | 420 | 1..7 | 51..414 | SERPIN |
| | Serpina3c | NP_032484.1 | 16625 | 417 | 1..22 | 51..41 | alpha-1- |
| | | | | | | | antitrypsin_like |
| | Serpina3f | NP_001161766.1 | 238393 | 445 | — | 40..405 | alpha-1- |
| | | | | | | | antitrypsin_like |
| | Serpina3g | NP_033277.2 | 20715 | 440 | — | 46..407 | SERPIN |
| | Serpina3i | NP_001186869.1 | 628900 | 408 | — | 46..407 | SERPIN |
| | Serpina3j | NP_001094942.1 | 238395 | 420 | 1..20 | 56..417 | SERPIN |
| | Serpina3k | NP_035588.2 | 20714 | 418 | 1..21 | 57..417 | SERPIN |
| | Serpma3m | NP_033279.2 | 20717 | 418 | 1..20 | 56..417 | SERPIN |
| | Serpina3n | NP_0332782 | 20716 | 418 | 1..20 | 51..414 | alpha-1- |
| | | NM_009252.2 | | | | | antitrypsin_like |
| rat | Serpina3n | NP_113719.1 | 24795 | 408 | — | 41..404 | alpha-1- |
| | | NM_031531.1 | | | | | antitrypsin_like |
| | Serpina3m | NP_001257911.1 | 299276 | 419 | 1..20 | 56..416 | SERPIN |
| Macaco | SERPINA3 | NP_001182279.1 | 574106 | 424 | 1..23 | 52..418 | alpha-1- |
| mulatta | | | | | | | antitrypsin_like |
| Pongo | SERPINA3 | NP_001126852.1 | 100173860 | 423 | 1..23 | 52..417 | alpha-1- |
| abelii | | | | | | | antitrypsin_like |
| Boa | SERPINA3 | NP_001075213.1 | 617667 | 411 | 1..24 | 46..405 | alpha-1- |
| taurus | | | | | | | antitrypsin_like |
| | SERPINA3-1 | NP_777193.2 | 286804 | 411 | 1..24 | 46..405 | alpha-1- |
| | | | | | | | antitrypsin_like |
| | SERP1NA3-2 | NP_001139773.1 | 100272170 | 411 | 1..24 | 46..405 | alpha-1- |
| | | | | | | | antitrypsin_like |
| | SERPINA3-3 | NP_001033293.1 | 615103 | 411 | 1..24 | 46..405 | alpha-1- |
| | | | | | | | antitrypsin_like |
| | SERPINA3-6 | NP_001139774.1 | 100272171 | 414 | 1..25 | 49..408 | alpha-1- |
| | | | | | | | antitrypsin_like |
| | SERPINA3-7 | NP_001012283.2 | 497203 | 417 | 1..25 | 49..411 | alpha-1- |
| | | | | | | | antitrypsin_like |
| | SERPINA3-8 | NP_001075181.1 | 505820 | 418 | 1..25 | 49..412 | alpha-1- |
| | | | | | | | antitrypsin_like |
| Sus | SERPINA3-2 | NP_998952.1 | 396686 | 415 | 1..22 | 50..412 | alpha-1- |
| scrofa | | | | | | | antitrypsin_like |

| | | | | | | | SEQ |
|---|---|---|---|---|---|---|---|
| | | NCBI | | | | | ID |
| | Gene | data | OrthoDB | Ref. 1 | Ref. 2 | Ref. 3 | NO. |
| human | SERPINA3 | | | | | | 1.29 |
| mouse | Serpina3a | | a3/a3n | homo | homo | | 2 |
| | | | ortholog | | | | |
| | Serpina3b | | a3/a3n | homo | homo | | 3 |
| | | | ortholog | | | | |
| | Serpina3c | homo | a3/a3n | homo | homo | | 4 |
| | | | ortholog | | | | |
| | Serpina3f | | a3/a3n | homo | homo | | 5 |
| | | | ortholog | | | | |
| | Serpina3g | | a3/a3n | homo | homo | | 6 |
| | | | ortholog | | | | |
| | Serpina3i | | a3/3n | | homo | | 7 |
| | | | ortholog | | | | |
| | Serpina3j | | a3/a3n | | homo | | 8 |
| | | | ortholog | | | | |
| | Serpina3k | homo | a3/a3n | homo | homo | | 9 |
| | | | ortholog | | | | |
| | Serpma3m | homo | a3/a3n | homo | homo | | 10 |
| | | | ortholog | | | | |
| | Serpina3n | homo | a3 | homo | homo | | 11.30 |
| | | | ortholog | | ortholog | | |
| rat | Serpina3n | homo | a3/a3n | | homo a3n | | 12.31 |
| | | | ortholog | | ortholog | | |
| | Serpina3m | | a3/a3n | | homo | | 13 |
| | | | ortholog | | | | |
| Macaco | SERPINA3 | homo a3 | a3 | | | | 14 |
| mulatta | | ortholog | ortholog | | | | |
| Pongo | SERPINA3 | a3 | a3 | | | | 15 |
| abelii | | ortholog | ortholog | | | | |
| Boa | SERPINA3 | homo | a3 | | | homo | 16 |
| taurus | | | ortholog | | | | |
| | SERPINA3-1 | homo | a3 | | homo | homo | 17 |
| | | | ortholog | | | | |
| | SERP1NA3-2 | | | | | homo | 18 |
| | SERPINA3-3 | homo | a3 | | | homo | 19 |
| | | | ortholog | | | | |
| | SERPINA3-6 | | | | | homo | 20 |
| | SERPINA3-7 | homo | a3 | | homo | homo | 21 |
| | | | ortholog | | | | |
| | SERPINA3-8 | homo | a3 | | | homo | 22 |
| | | | ortholog | | | | |
| Sus | SERPINA3-2 | | a3 | | homo | homo | 23 |
| scrofa | | | ortholog | | | | |

homo: human SERPINA3 homolog
a3 ortholog: human SERPINA3 ortholog
a3n ortholog: mouse Serpina3n ortholog
NCBI data: https://www.ncbi.nlm.mh.gov/
OrthoDB: https://www.orthodb.org/
Ref. 1: Heit et al. Human Genomics. 7: 22.2013
Ref. 2: Horvath et al. J. Biol. Chem. 280. 43168-43178.2005
Ref. 3: Pelissier et al. BMC Genomics. 9: 151, 2008

Table 1 shows human SERPINA3 gene homologs (homo), human SERPINA3 gene orthologs (a3 ortholog), and mouse SERPINA3N gene orthologs (a3n ortholog) found in the NCBI database (NCBI data: https://www.ncbi.nlm.nih.gov/), an ortholog database (OrthoDB: https://www.orthodb.org/), or literatures (Ref. 1: Heit et al., Human Genomics, 7: 22, 2013; Ref. 2: Horvath et al., J. Biol. Chem, 280, 43168-43178, 2005; Ref 3: Pelissier et al., BMC Genomics, 9: 151, 2008). Table 1 shows the ID of each gene (NCBI Reference Sequence; Entrez gene ID), the amino acid length of the encoded protein (amino acids), the amino acid position of the signal peptide (signal), and the conserved region (region) and its name (region name).

In an embodiment, serpin A3 is a protein encoded by the human SERPINA3 gene or an ortholog thereof. The protein encoded by the ortholog of the human SERPINA3 gene can be, but not limited to, a protein encoded by the gene shown as an ortholog in Table 1.

In another embodiment, serpin A3 is a protein encoded by the human SERPINA3 gene, or a protein encoded by a homolog of the human SERPINA3 gene and having an al-antitrypsin-like structure. The protein encoded by a homolog of the human SERPINA3 gene and having an al-antitrypsin-like structure can be, but not limited to, a protein encoded by the gene shown to contain an al-antitrypsin-like structure in Table 1.

In a further embodiment, serpin A3 is a protein encoded by the human SERPINA3 gene, mouse SERPINA3N gene, or rat SERPINA3N gene, i.e., human serpin A3, mouse serpin A3N, or rat serpin A3N.

Serpin A3 can be a protein consisting of an amino acid sequence having a total length of about 400 to 450 residues, although it varies depending on the species, and it is known to exist extracellularly as a secretory protein in vivo. A protein consisting of a full-length amino acid sequence encoded by a serpin gene is active, and also some C-terminal polypeptide fragments generated by cleavage of the N-terminus of full-length proteins are known to function as mature proteins.

In the present disclosure, “serpin A3” includes a full-length serpin A3 and a mature serpin A3. The full-length serpin A3 means a protein consisting of a full-length amino acid sequence encoded by a serpin A3 gene. The mature serpin A3 means a C-terminal polypeptide that is confirmed or suggested to generate when a full-length serpin A3 is processed by a protease and has a biological activity. The mature serpin A3 can be, for example, a C-terminal polypeptide of a full-length serpin A3 that lacks its N-terminal signal peptide.

For example, serpin A3 can be a polypeptide selected from a) to g) below:

a) a polypeptide comprising or consisting of an amino acid sequence of a full-length or mature serpin A3,
b) a polypeptide comprising an amino acid sequence of a mature serpin A3 and consisting of a partial amino acid sequence of a full-length serpin A3,
c) a polypeptide comprising or consisting of a partial amino acid sequence of a full-length or mature serpin A3 and being functionally equivalent to the full-length or mature serpin A3,
d) a polypeptide comprising or consisting of an amino acid sequence that differs from an amino acid sequence of a full-length or mature serpin A3 in that one or more, for example 1 to 10, 1 to 5, 1 to 3 or 1 or 2 amino acids are substituted, deleted, inserted or added, and being functionally equivalent to the full-length or mature serpin A3,
e) a polypeptide comprising or consisting of an amino acid sequence having about 80% or more, for example about 85% or more, about 90% or more, about 95% or more, about 96% or more, about 97% or more, about 98% or more or about 99% or more sequence identity with an amino acid sequence of a full-length or mature serpin A3 and being functionally equivalent to the full-length or mature serpin A3,
f) a polypeptide encoded by a DNA that hybridizes to a nucleic acid sequence encoding a full-length or mature serpin A3 under a stringent condition and being functionally equivalent to the full-length or mature serpin A3, and
g) a polypeptide encoded by a nucleic acid sequence having about 70% or more, for example about 75% or more, about 80% or more, about 85% or more, about 90% or more, about 95% or more, about 96% or more, about 97% or more, about 98% or more, or about 99% or more sequence identity with a nucleic acid sequence encoding a full-length or mature serpin A3 and being functionally equivalent to the full-length or mature serpin A3.

The phrase being “functionally equivalent” to a full-length or mature serpin A3 means that it has a biological activity of the same nature as the protein, and the phrase “of the same nature” here means being the same in qualitative evaluation. Examples of biological activities of a full-length or mature serpin A3 in the present disclosure include, for example, promotion of growth or suppression of decrease of mesenchymal stem cells, as well as inhibitory activity on a serine protease, for example, inhibitory activity on one or more serine proteases selected from chymotrypsin, trypsin, elastase, cathepsin G, and chymase. In an embodiment, a polypeptide being functionally equivalent to a full-length or mature serpin A3 is a polypeptide having the activity of promoting growth or suppressing decrease of mesenchymal stem cells.

In the present disclosure, the identity of amino acid sequences or nucleic acid sequences means the degree of sequence matching between polypeptides or polynucleotides, and it is determined by comparing two sequences optimally aligned (aligned so that amino acids or nucleotides maximally match) over the sequence region to be compared. The numerical value of the sequence identity (%) is calculated by identifying the same amino acids or nucleotides present in both sequences to determine the number of matching sites, dividing the number of matching sites by the total number of amino acids or nucleotides in the sequence region to be compared, and multiplying the obtained value by 100. Examples of algorithms for obtaining optimal alignment and sequence identity include various algorithms commonly available to those of skill in the art (e.g., BLAST algorithm, FASTA algorithm). The sequence identity can be determined, for example, by using a sequence analysis software such as BLAST or FASTA.

With respect to the hybridization under a stringent condition, such hybridization can be performed according to conventional methods described in literatures such as Molecular Cloning, T. Maniatis et al., CSH Laboratory (1983), for example. The “stringent condition” includes a condition comprising hybridizing in a solution containing 6×SSC (wherein a solution containing 1.5 M NaCl and 0.15 M trisodium citrate is called 10×SSC) and 50% formamide at 45° C. and then washing with 2×SSC at 50° C. (Molecular Biology, John Wiley & Sons, N.Y. (1989), 6.3.1-6.3.6), and conditions that result in stringency equivalent thereto.

The representative nucleic acid sequences encoding human serpin A3, mouse serpin A3N, and rat serpin A3N, and the amino acid sequences of these full-length and mature proteins are shown below.

Human serpin A3 full-length protein (NP_001076.2)
(SEQ ID NO: 1)
MERMLPLLALGLLAAGFCPAVLCHPNSPLDEENLTQENQDRGTHVDLGLASANVDFAFS
LYKQLVLKAPDKNVIFSPLSISTALAFLSLGAHNTTLTEILKGLKFNLTETSEAEIHQS
FQHLLRTLNQSSDELQLSMGNAMFVKEQLSLLDRFTEDAKRLYGSEAFATDFQDSAAAK
KLINDYVKNGTRGKITDLIKDLDSQTMMVLVNYIFFKAKWEMPFDPQDTHQSRFYLSKK
KWVMVPMMSLHHLTIPYFRDEELSCTVVELKYTGNASALFILPDQDKMEEVEAMLLPET
LKRWRDSLEFREIGELYLPKFSISRDYNLNDILLQLGIEEAFTSKADLSGITGARNLAV
SQVVHKAVLDVFEEGTEASAATAVKITLLSALVETRTIVRFNRPFLMIIVPTDTQNIFF
MSKVTNPKQA
Human serpin A3 mature protein (24-423 of SEQ ID NO: 1)
(SEQ ID NO: 24)
HPNSPLDEENLTQENQDRGTHVDLGLASANVDFAFSLYKQLVLKAPDKNVIFSPLSIST
ALAFLSLGAHNTTLTEILKGLKFNLTETSEAEIHQSFQHLLRTLNQSSDELQLSMGNAM
FVKEQLSLLDRFTEDAKRLYGSEAFATDFQDSAAAKKLINDYVKNGTRGKITDLIKDLD
SQTMMVLVNYIFFKAKWEMPFDPQDTHQSRFYLSKKKWVMVPMMSLHHLTIPYFRDEEL
SCTVVELKYTGNASALFILPDQDKMEEVEAMLLPETLKRWRDSLEFREIGELYLPKFSI
SRDYNLNDILLQLGIEEAFTSKADLSGITGARNLAVSQVVHKAVLDVFEEGTEASAATA
VKITLLSALVETRTIVRFNRPFLMIIVPTDTQNIFFMSKVTNPKQA
Human serpin A3 mature protein (26-423 of SEQ ID NO: 1)
(SEQ ID NO: 25)
NSPLDEENLTQENQDRGTHVDLGLASANVDFAFSLYKQLVLKAPDKNVIFSPLSISTAL
AFLSLGAHNTTLTEILKGLKFNLTETSEAEIHQSFQHLLRTLNQSSDELQLSMGNAMFV
KEQLSLLDRFTEDAKRLYGSEAFATDFQDSAAAKKLINDYVKNGTRGKITDLIKDLDSQ
TMMVLVNYIFFKAKWEMPFDPQDTHQSRFYLSKKKWVMVPMMSLHHLTIPYFRDEELSC
TVVELKYTGNASALFILPDQDKMEEVEAMLLPETLKRWRDSLEFREIGELYLPKFSISR
DYNLNDILLQLGIEEAFTSKADLSGITGARNLAVSQVVHKAVLDVFEEGTEASAATAVK
ITLLSALVETRTIVRFNRPFLMIIVPTDTQNIFFMSKVTNPKQA
Mouse serpin A3N full-length protein (NP_033278.2)
(SEQ ID NO: 11)
MAFIAALGLLMAGICPAVLCFPDGTLGMDAAVQEDHDNGTQLDSLTLASINTDFAFSLY
KELVLKNPDKNIVFSPLSISAALAVMSLGAKGNTLEEILEGLKFNLTETSEADIHQGFG
HLLQRLNQPKDQVQISTGSALFIEKRQQILTEFQEKARALYQAEAFTADFQQPRQAKKL
INDYVRKQTQGMIKELVSDLDKRTLMVLVNYIYFKAKWKVPFDPLDTFKSEFYAGKRRP
VIVPMMSMEDLTTPYFRDEELFCTVVELKYTGNASAMFILPDQGKMQQVEASLQPETLR
KWKNSLKPRMIDELHLPKFSISTDYSLEDVLSKLGIREVFSTQADLSAITGTKDLRVSQ
VVHKAVLDVAETGTEAAAATGVKFVPMSAKLYPLTVYFNRPFLIMIFDTETEIAPFIAK
IANPK
Mouse serpin A3N mature protein (21-418 of SEQ ID NO: 11)
(SEQ ID NO: 26)
FPDGTLGMDAAVQEDHDNGTQLDSLTLASINTDFAFSLYKELVLKNPDKNIVFSPLSIS
AALAVMSLGAKGNTLEEILEGLKFNLTETSEADIHQGFGHLLQRLNQPKDQVQISTGSA
LFIEKRQQILTEFQEKARALYQAEAFTADFQQPRQAKKLINDYVRKQTQGMIKELVSDL
DKRTLMVLVNYIYFKAKWKVPFDPLDTFKSEFYAGKRRPVIVPMMSMEDLTTPYFRDEE
LFCTVVELKYTGNASAMFILPDQGKMQQVEASLQPETLRKWKNSLKPRMIDELHLPKFS
ISTDYSLEDVLSKLGIREVFSTQADLSAITGTKDLRVSQVVHKAVLDVAETGTEAAAAT
GVKFVPMSAKLYPLTVYFNRPFLIMIFDTETEIAPFIAKIANPK
Mouse serpin A3N full-length protein
(SEQ ID NO: 27)
MAFIAALGLLMAGICPAVLCFPDGTLGMDAAVQEDHDNGTQLDSLTLASINTDFAFSLY
KELVLKNPDKNIVFSPLSISAALAVMSLGAKGNTLEEILEGLKFNLTETSEADIHQGFG
HLLQRLNQPKDQVQISTGSALFIEKRQQILTEFQEKAKTLYQAEAFTADFQQPRQAKKL
INDYVRKQTQGMIKELVSDLDKRTLMVLVNYIYFKAKWKVPFDPLDTFKSEFYAGKRRP
VIVPMMSMEDLTTPYFRDEELSCTVVELKYTGNASALFILPDQGRMQQVEASLQPETLR
KWKNSLKPRMIDELHLPKFSISTDYSLEDVLSKLGIREVFSTQADLSAITGTKDLRVSQ
VVHKAVLDVAETGTEAAAATGVKFVPMSAKLYPLTVYFNRPFLIMIFDTETEIAPFIAK
IANPK
Mouse serpin A3N mature protein (21-418 of SEQ ID NO: 27)
(SEQ ID NO: 28)
FPDGTLGMDAAVQEDHDNGTQLDSLTLASINTDFAFSLYKELVLKNPDKNIVFSPLSIS
AALAVMSLGAKGNTLEEILEGLKFNLTETSEADIHQGFGHLLQRLNQPKDQVQISTGSA
LFIEKRQQILTEFQEKAKTLYQAEAFTADFQQPRQAKKLINDYVRKQTQGMIKELVSDL
DKRTLMVLVNYIYFKAKWKVPFDPLDTFKSEFYAGKRRPVIVPMMSMEDLTTPYFRDEE
LSCTVVELKYTGNASALFILPDQGRMQQVEASLQPETLRKWKNSLKPRMIDELHLPKFS
ISTDYSLEDVLSKLGIREVFSTQADLSAITGTKDLRVSQVVHKAVLDVAETGTEAAAAT
GVKFVPMSAKLYPLTVYFNRPFLIMIFDTETEIAPFIAKIANPK
Rat serpin A3N full-length protein (NP_113719.1)
(SEQ ID NO: 12)
MDGIGSALLSFPDCILGEDTLFHEDQDKGTQLDSLTLASINTDFAFSLYKKLALRNPHK
NVVFSPLSISAALAVVSLGAKGSSMEEILEGLKFNLTETPETEIHRGFGHLLQRLSQPR
DEIQISTGNALFIEKRLQVLAEFQEKAKALYQAEAFTADFQQSREAKKLINDYVSKQTQ
GKIQGLITNLAKKTSMVLVNYIYFKGKWKVPFDPRDTFQSEFYSGKRRSVKVPMMKLED
LTTPYVRDEELNCTVVELKYTGNASALFILPDQGKMQQVEASLQPETLRRWKDSLRPSM
IDELYLPKFSISADYNLEDVLPELGIKEVFSTQADLSGITGDKDLMVFQVVHKAVLDVA
ETGTEAAAATGVKFVPMSAKLDPLIIAFDRPFLMIISDTETAIAPFLAKIFNPK
Human serpin A3 nucleic acid sequence (NM_001085.5)
(SEQ ID NO: 29)
ATGGAGAGAATGTTACCTCTCCTGGCTCTGGGGCTCTTGGCGGCTGGGTTCTGCCCTGC
TGTCCTCTGCCACCCTAACAGCCCACTTGACGAGGAGAATCTGACCCAGGAGAACCAAG
ACCGAGGGACACACGTGGACCTCGGATTAGCCTCCGCCAACGTGGACTTCGCTTTCAGC
CTGTACAAGCAGTTAGTCCTGAAGGCCCCTGATAAGAATGTCATCTTCTCCCCACTGAG
CATCTCCACCGCCTTGGCCTTCCTGTCTCTGGGGGCCCATAATACCACCCTGACAGAGA
TTCTCAAAGGCCTCAAGTTCAACCTCACGGAGACTTCTGAGGCAGAAATTCACCAGAGC
TTCCAGCACCTCCTGCGCACCCTCAATCAGTCCAGCGATGAGCTGCAGCTGAGTATGGG
AAATGCCATGTTTGTCAAAGAGCAACTCAGTCTGCTGGACAGGTTCACGGAGGATGCCA
AGAGGCTGTATGGCTCCGAGGCCTTTGCCACTGACTTTCAGGACTCAGCTGCAGCTAAG
AAGCTCATCAACGACTACGTGAAGAATGGAACTAGGGGGAAAATCACAGATCTGATCAA
GGACCTTGACTCGCAGACAATGATGGTCCTGGTGAATTACATCTTCTTTAAAGCCAAAT
GGGAGATGCCCTTTGACCCCCAAGATACTCATCAGTCAAGGTTCTACTTGAGCAAGAAA
AAGTGGGTAATGGTGCCCATGATGAGTTTGCATCACCTGACTATACCTTACTTCCGGGA
CGAGGAGCTGTCCTGCACCGTGGTGGAGCTGAAGTACACAGGCAATGCCAGCGCACTCT
TCATCCTCCCTGATCAAGACAAGATGGAGGAAGTGGAAGCCATGCTGCTCCCAGAGACC
CTGAAGOGGTGGAGAGACTCTCTGGAGTTCAGAGAGATAGGTGAGCTCTACCTGCCAAA
GTTTTCCATCTCGAGGGACTATAACCTGAACGACATACTTCTCCAGCTGGGCATTGAGG
AAGCCTTCACCAGCAAGGCTGACCTGTCAGGGATCACAGGGGCCAGGAACCTAGCAGTC
TCCCAGGTGGTCCATAAGGCTGTGCTTGATGTATTTGAGGAGGGCACAGAAGCATCTGC
TGCCACAGCAGTCAAAATCACCCTCCTTTCTGCATTAGTGGAGACAAGGACCATTGTGC
GTTTCAACAGGCCCTTCCTGATGATCATTGTCCCTACAGACACCCAGAACATCTTCTTC
ATGAGCAAAGTCACCAATCCCAAGCAAGCCTAG
Mouse serpin A3N nucleic acid sequence (NM_009252.2)
(SEQ ID NO: 30)
ATGGCCTTCATTGCAGCTCTGGGGCTCTTGATGGCTGGGATCTGCCCTGCTGTCCTCTG
CTTCCCAGATGGCACGTTGGGAATGGATGCTGCAGTCCAAGAAGACCATGACAATGGGA
CACAACTGGACAGTCTCACATTGGCCTCCATCAACACTGACTTTGCCTTCAGCCTCTAC
AAGGAGCTGGTTTTGAAGAATCCAGATAAAAATATTGTCTTCTCCCCACTTAGCATCTC
AGCGGCCTTGGCCGTCATGTCCCTGGGAGCAAAGGGCAACACCCTGGAAGAGATTCTAG
AAGGTCTCAAGTTCAATCTTACAGAGACCTCTGAGGCAGACATCCACCAGGGCTTTGGG
CACCTCCTACAGAGGCTCAACCAGCCAAAGGACCAGGTACAGATCAGCACGGGTAGTGC
CCTGTTTATTGAAAAGCGCCAGCAGATCCTGACAGAATTCCAGGAGAAGGCAAGGGCTC
TGTACCAGGCTGAGGCCTTCACAGCAGACTTCCAGCAGCCTCGTCAGGCCAAAAAGCTC
ATCAATGACTATGTGAGGAAACAGACCCAGGGGATGATCAAGGAACTGGTCTCAGACCT
GGATAAAAGGACATTGATGGTGCTGGTGAATTATATCTACTTTAAAGCCAAATGGAAGG
TGCCCTTTGACCCTCTTGACACGTTCAAGTCTGAGTTCTACGCGGGCAAGAGGAGGCCC
GTGATAGTGCCCATGATGAGCATGGAGGACCTGACCACACCCTACTTCCGAGATGAGGA
GCTTTTCTGCACTGTGGTGGAGCTGAAGTACACAGGAAATGCCAGTGCCATGTTCATCC
TCCCTGACCAGGGCAAGATGCAGCAGGTGGAAGCCAGCTTGCAACCAGAGACCCTGAGG
AAGTGGAAGAATTCTCTGAAACCCAGGATGATAGATGAGCTCCACCTGCCCAAGTTCTC
CATCTCCACCGACTACAGCCTGGAGGATGTCCTTTCAAAGCTGGGCATCAGGGAAGTCT
TCTCCACACAGGCTGACCTGTCTGCAATCACAGGAACCAAGGATCTGAGAGTCTCTCAG
GTGGTCCACAAGGCTGTGCTGGACGTGGCTGAGACAGGCACAGAAGCAGCTGCTGCCAC
TGGAGTCAAATTTGTCCCAATGTCTGCGAAACTGTACCCTCTGACTGTATATTTCAATC
GGCCTTTCCTGATAATGATCTTTGACACAGAAACTGAAATTGCCCCCTTTATAGCCAAG
ATAGCCAACCCCAAATGA
Rat serpin A3N nucleic acid sequence (NM_031531.1)
(SEQ ID NO: 31)
ATGGATGGGATCGGCTCTGCTCTCCTCTCCTTCCCAGATTGCATACTGGGAGAGGACAC
TCTATTCCATGAAGACCAAGACAAGGGGACACAACTGGACAGTCTCACATTGGCCTCCA
TCAATACTGACTTTGCCTTCAGCCTCTACAAGAAGCTGGCTTTGAGGAATCCACATAAA
AATGTTGTCTTCTCCCCACTTAGCATCTCAGCCGCCTTGGCCGTCGTGTCCCTGGGAGC
AAAGGGCAGCAGCATGGAAGAGATTCTAGAAGGTCTCAAGTTCAATCTCACAGAGACCC
CTGAGACAGAAATCCACCGGGGCTTTGGACACCTCCTCCAGAGGCTCAGCCAGCCAAGG
GACGAGATACAGATCAGTACAGGCAATGCCCTGTTTATTGAAAAACGCCTTCAGGTCCT
GGCAGAGTTCCAGGAGAAGGCAAAGGCTCTGTACCAAGCTGAGGCCTTCACAGCTGATT
TCCAGCAGTCTCGTGAGGCCAAAAAGCTCATCAATGACTATGTGAGTAAACAGACCCAG
GGGAAGATCCAGGGACTGATCACAAACCTAGCTAAGAAGACATCCATGGTACTGGTGAA
TTACATCTACTTTAAAGGCAAATGGAAGGTGCCTTTTGACCCTCGGGACACATTCCAGT
CTGAGTTCTACTCTGGCAAAAGGAGGTCTGTGAAAGTGCCCATGATGAAGCTTGAGGAC
CTGACCACACCCTACGTCCGGGATGAGGAGCTGAACTGCACTGTTGTGGAGCTGAAGTA
CACAGGAAATGCCAGCGCCCTGTTTATCCTCCCTGACCAGGGCAAGATGCAGCAGGTGG
AAGCCAGCTTGCAACCAGAGACCCTGAGGAGATGGAAGGACTCTCTCAGGCCCAGCATG
ATAGATGAGCTCTACCTGCCCAAGTTCTCCATCTCTGCTGACTACAACCTGGAGGACGT
CCTTCCAGAGCTGGGCATCAAAGAAGTCTTCTCCACACAGGCTGACCTGTCTGGGATCA
CAGGGGATAAGGACCTGATGGTCTTTCAGGTGGTCCACAAGGCTGTTCTGGATGTGGCT
GAGACAGGCACAGAAGCAGCCGCTGCCACAGGGGTCAAATTTGTTCCAATGTCTGCAAA
ACTGGACCCTCTGATTATAGCTTTCGACCGGCCTTTCCTGATGATTATCTCTGACACAG
AAACTGCAATAGCTCCCTTTTTGGCCAAGATATTTAACCCCAAATGA

In an embodiment, the full-length serpin A3 comprises or consists of an amino acid sequence selected from SEQ ID NOs: 1 to 23 and 27. In a further embodiment, the full length serpin A3 comprises or consists of an amino acid sequence selected from SEQ ID NOs: 1, 2 to 17, 19, 21 to 23 and 27. In a further embodiment, the full-length serpin A3 comprises or consists of an amino acid sequence selected from SEQ ID NOs: 1, 4, 5, 11, 12, 14 to 23 and 27. In a further embodiment, the full-length serpin A3 comprises or consists of the amino acid sequence of SEQ ID NO: 1, 11, 12, or 27.

In an embodiment, the mature serpin A3 comprises or consists of an amino acid sequence selected from SEQ ID NOs: 1 to 23 excluding its signal peptide, or comprises or consists of the amino acid sequence of SEQ ID NO: 24, 25, 26 or 28. In a further embodiment, the mature serpin A3 comprises or consists of an amino acid sequence selected from SEQ ID NOs: 1, 2 to 17, 19, and 21 to 23 excluding its signal peptide, or comprises or consists of the amino acid sequence of SEQ ID NO: 24, 25, 26 or 28. In a further embodiment, the mature serpin A3 comprises or consists of an amino acid sequence selected from SEQ ID NOs: 1, 4, 5, 11, 12, and 14 to 23 excluding its signal peptide, or comprises or consists of the amino acid sequence of SEQ ID NO: 24, 25, 26 or 28.

In a further embodiment, the mature serpin A3 comprises or consists of an amino acid sequence selected from SEQ ID NOs: 1 to 4, 8 to 11, and 13 to 23 excluding the signal peptide shown in Table 1, or comprises or consists of the amino acid sequence of SEQ ID NO: 24, 25, 26 or 28. In a further embodiment, the mature serpin A3 comprises or consists of an amino acid sequence selected from SEQ ID NOs: 1, 2 to 4, 8 to 11, 13 to 17, 19, and 21 to 23 excluding the signal peptide shown in Table 1, or comprises or consists of the amino acid sequence of SEQ ID NO: 24, 25, 26 or 28. In a further embodiment, the mature serpin A3 comprises or consists of an amino acid sequence selected from SEQ ID NOs: 1, 4, 11, and 14 to 23 excluding the signal peptide shown in Table 1, or comprises or consists of the amino acid sequence of SEQ ID NO: 24, 25, 26 or 28.

In a further embodiment, the mature serpin A3 comprises or consists of the amino acid sequence of SEQ ID NO: 24, 25, 26 or 28.

In the present disclosure, the full-length or mature serpin A3 or a polypeptide being functionally equivalent thereto may be a modified polypeptide. Examples of such modifications include tag addition, sugar chain addition, partial sugar chain addition, and sugar chain non-formation, wherein the sugar chain may be a natural or modified one.

The full-length or mature serpin A3 or a polypeptide being functionally equivalent thereto may be obtained by any method. It can be prepared, for example, by recombinant expression (using mammalian cells, yeast, Escherichia coli, or insect cells, for example) or by synthesis using a cell-free system, and it can also be a purchasable commercial product.

Inflammatory bowel disease (herein also referred to as IBD) refers to a chronic or relapsing-remitting inflammatory disease of the intestinal tract. IBD includes ulcerative colitis and Crohn's disease. The examples of this disclosure demonstrate that serpin A3 suppresses decrease of mesenchymal stem cells in the bone marrow in dextran sodium sulfate (DSS)-induced IBD model mice. Therefore, serpin A3 or a composition comprising the same can be used to suppress decrease of mesenchymal stem cells caused by IBD, or to treat or prevent IBD.

The treating IBD includes inducing remission, maintaining remission, and suppressing relapse. In an embodiment, serpin A3 or a composition comprising the same is used to maintain remission or to suppress relapse. In the present disclosure, subjects suffering from IBD (also referred to as IBD patients) include IBD patients in the active phase and those in the remission phase.

For the treatment of IBD, a purified and formulated serpin A3 polypeptide may be used, or a tissue or cell that secretes serpin A3 or secreted material or culture supernatant containing serpin A3 from such a tissue or cell may be used.

Serpin A3 or a composition comprising the same is administered to a subject in an amount capable of exerting a desired effect (herein referred to as “an effective amount”). The dose is appropriately determined depending on factors such as age, body weight, and health condition of the subject. For example, as an amount of serpin A3, the dose can be selected in the range of 0.0000001 mg to 1000 mg per kg of body weight per administration. Alternatively, the dose can be selected in the range of 0.00001 to 100000 mg/body per subject. However, the dose is not limited to these doses. Serpin A3 or a composition comprising the same may be administered once daily or in multiple doses (e.g., 2, 3 or 4 times) per day, and may be administered at an interval(s) of one or several days (e.g., 2, 3, 4, 5 or 6 days), one or several weeks (e.g., 2, 3, 4, 5 or 6 weeks), one or several months (e.g., 2, 3, 4, 5 or 6 months). The duration of administration is also not limited, and may be one or several days (e.g., 2, 3, 4, 5 or 6 days), one or several weeks (e.g., 2, 3, 4, 5 or 6 weeks), one or several months (e.g., 2, 3, 4, 5 or 6 months).

Serpin A3 or a composition comprising the same can be administered systemically or topically. Examples of administration methods include oral administration, intravenous administration, intramuscular administration, subcutaneous administration, intracutaneous administration, intraperitoneal administration, and intrathecal administration. In an embodiment, serpin A3 or a composition comprising the same is administered intravenously or intrathecally.

When serpin A3 or a composition comprising the same is used in mesenchymal stem cell culturing, it may be added to the medium at a final concentration of serpin A3 of 1 pg/mL to 1 mg/mL, 10 pg/mL to 100 μg/mL, 100 pg/mL to 10 μg, 1 ng/mL to 1 μg/mL, 1 ng/mL to 100 ng/mL, or 1 ng/mL to 10 ng/mL.

The composition of the present disclosure can be formulated according to conventional methods (for example, according to Remington's Pharmaceutical Science, latest edition, Mark Publishing Company, Easton, U.S.A). It may comprise a pharmaceutically acceptable carrier in addition to an active ingredient. Examples of pharmaceutically acceptable carriers include surfactants, excipients, colorants, flavoring agents, preservatives, stabilizers, buffers, suspending agents, tonicity agents, binders, disintegrants, lubricants, fluidity promoters, and taste masking agents. For example, the pharmaceutically acceptable carrier may be light anhydrous silicic acid, lactose, crystalline cellulose, mannitol, starch, carmellose calcium, carmellose sodium, hydroxypropyl cellulose, hydroxypropylmethyl cellulose, polyvinyl acetal diethylaminoacetate, polyvinylpyrrolidone, gelatin, a medium chain fatty acid triglyceride, polyoxyethylene hydrogenated castor oil 60, sucrose, carboxymethyl cellulose, corn starch, or an inorganic salt. Further, when the treatment of IBD is intended and the composition comprises cells, the pharmaceutically acceptable carrier not only may be any of the above, but also may be water, medium, physiological saline, an isotonic solution containing glucose, D-sorbitol, D-mannose, or D-mannitol, or phosphate buffered saline (PBS).

The dosage form of the composition is, but not limited to, a preparation for oral or parenteral administration, and it can be an injection. Examples of injections include solution injections, suspension injections, emulsion injections, and injections to be prepared before use. The composition may be frozen and may contain a cryoprotectant such as DMSO, glycerol, polyvinylpyrrolidone, polyethylene glycol, albumin, dextran, or sucrose.

Serpin A3 or a composition comprising the same may be used as an additive for a medium used for mesenchymal stem cell culturing. The medium additive may be provided in any form, e.g., as a solid such as granule, powder, or tablet, a liquid such as solution, suspension, or emulsion, or a capsule containing solid, semi-solid or liquid content, although it is not limited thereto. The medium additive can be used for culturing adherent cells containing mesenchymal stem cells, for example, when added to a common culture medium for animal cells including mesenchymal stem cells. The medium is not limited as long as it can be used for culturing mesenchymal stem cells, and examples thereof include MEM, MEMα, DMEM, GMEM, RPMI 1640, and MesenCult™ (STEMCELL Technologies). Serpin A3 can be added to/contained in any medium as exemplified above for culturing adherent cells including mesenchymal stem cells. Any additional component may be added to the medium as long as it does not inhibit growth of mesenchymal stem cells.

Exemplary embodiments of the present invention are described below.

[1] A composition for promoting growth or suppressing decrease of mesenchymal stem cells comprising serpin A3.
[2] The composition according to item 1, wherein the mesenchymal stem cells are colony-forming mesenchymal stem cells.
[3] The composition according to item 1 or 2, wherein the mesenchymal stem cells are bone marrow mesenchymal stem cells.
[4] The composition according to any one of items 1 to 3, wherein the composition is administered to a subject.
[5] The composition according to item 4, wherein the subject suffers from inflammatory bowel disease.
[6] The composition according to any one of items 1 to 5, wherein the decrease of mesenchymal stem cells is caused by inflammatory bowel disease.
[7] The composition according to any one of items 1 to 3, wherein the composition is used in mesenchymal stem cell culturing.
[8] A composition for treating inflammatory bowel disease comprising serpin A3.
[9] The composition according to any one of items 1 to 8, wherein the serpin A3 is selected from the group consisting of:
a) a polypeptide comprising or consisting of an amino acid sequence of a full-length or mature serpin A3,
b) a polypeptide comprising an amino acid sequence of a mature serpin A3 and consisting of a partial amino acid sequence of a full-length serpin A3,
c) a polypeptide comprising or consisting of a partial amino acid sequence of a full-length or mature serpin A3 and having an activity of promoting growth or suppressing decrease of mesenchymal stem cells,
d) a polypeptide comprising or consisting of an amino acid sequence that differs from an amino acid sequence of a full-length or mature serpin A3 in that 1 to 10 amino acids are substituted, deleted, inserted or added, and having an activity of promoting growth or suppressing decrease of mesenchymal stem cells,
e) a polypeptide comprising or consisting of an amino acid sequence having about 90% or more sequence identity with an amino acid sequence of a full-length or mature serpin A3 and having an activity of promoting growth or suppressing decrease of mesenchymal stem cells,
f) a polypeptide encoded by a DNA that hybridizes to a nucleic acid sequence encoding a full-length or mature serpin A3 under a stringent condition and having an activity of promoting growth or suppressing decrease of mesenchymal stem cells, and
g) a polypeptide encoded by a nucleic acid sequence having about 90% or more sequence identity with a nucleic acid sequence encoding a full-length or mature serpin A3 and having an activity of promoting growth or suppressing decrease of mesenchymal stem cells.
[10] The composition according to item 9, wherein the serpin A3 is the polypeptide of a) or b).
[11] The composition according to item 9 or 10, wherein the full-length serpin A3 comprises the amino acid sequence of SEQ ID NO: 1, 11, 12, or 27.

[12] The composition according to any one of items 9 to 11, wherein the mature serpin A3 comprises the amino acid sequence of SEQ ID NO: 24, 25, 26 or 28.

[13] The composition according to any one of items 9 to 12, wherein the nucleic acid sequence encoding a full-length serpin A3 comprises the amino acid sequence of SEQ ID NO: 29, 30, or 31.
[14] The composition according to any one of items 1 to 13, wherein the serpin A3 is human serpin A3, mouse serpin A3N, or rat serpin A3N.
[15] Serpin A3 for use in promoting growth or suppressing decrease of mesenchymal stem cells.
[16] Serpin A3 for use in the treatment of inflammatory bowel disease.
Use of serpin A3 for the manufacture of a medicament for use in promoting growth or suppressing decrease of mesenchymal stem cells.
[17] Use of serpin A3 for the manufacture of a medicament for use in the treatment of inflammatory bowel disease.
[18] A method for promoting growth or suppressing decrease of mesenchymal stem cells, comprising administering serpin A3 to a subject.
[19] A method for treating inflammatory bowel disease, comprising administering serpin A3 to a subject.

All the references cited in this disclosure are herein incorporated by reference. All the above descriptions are non-limiting descriptions and can be modified as long as they do not depart from the scope of the invention as defined in the appended claims. Also, the following examples are all non-limiting examples and are provided solely to illustrate the present invention.

### EXAMPLES

### 1. Preparation of IBD Model Mouse

Drinking water containing 1.5 wt/vol % of dextran sulfate sodium salt (DSS) (36 to 50 kDa; MP Biomedicals, catalog number: 160110) was prepared and filtered through a 0.45 μm cellulose acetate membrane. The 1.5 wt/vol % DSS drinking water thus prepared was administered to 8-week-old C57BL/6J male mice (3 mice) for 6 days to induce colitis. From the 6th day, normal drinking water containing no DSS was administered. The body weight of the mice was measured on each day during the experimental period, and the body weight change was examined with the body weight on the start date of the experiment (start date of DSS administration) as 100%. For comparison, the body weight change of healthy mice (wild-type mice) bred without DSS administration was also examined. The p-value was calculated by two-way ANOVA.

Also, in an experiment in which mice were allowed to take DSS in the same manner as above, the large intestine was collected from the mice on the 3rd, 6th, 9th, 12th, and 15th days from the start date of DSS administration (day 0), and a large intestine image was taken with a camera. Then, the colon length was calculated from the image thus obtained using ImageJ software. The p-value was calculated by two-way ANOVA.

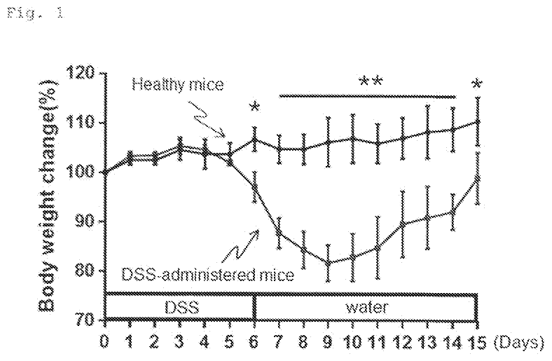

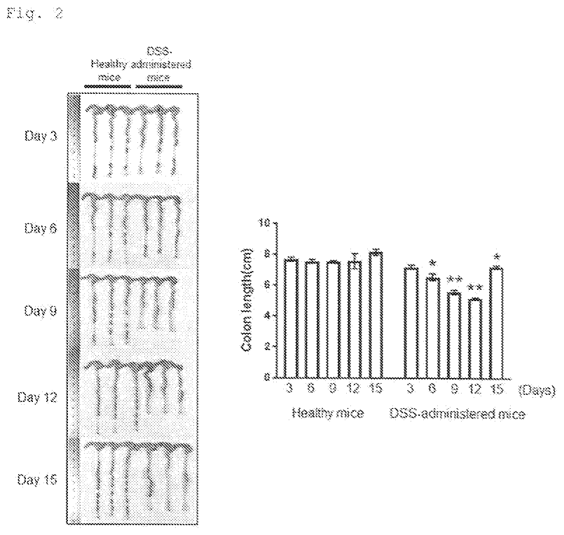

A significant weight loss was observed in the DSS-administered mice as compared with the healthy mice (FIG. 1). Also, on the 6th, 9th, 12th, and 15th days from the start date of DSS administration, the colon length of the DSS-administered mice was significantly decreased as compared with the healthy mice (FIG. 2). These results confirmed that the administration of 1.5 wt/vol % DSS solution induced colitis and could prepare IBD model mice.

### 2. Decrease of Colony-Forming Cells in Bone Marrow Cells of IBD Model Mice

Bone marrow cells were collected from the femurs of healthy mice and DSS-administered mice on the 3th, 6th, 9th, 12th, and 15th days from the start of the experiment (start of DSS administration). DSS was administered in the same manner as in section 1 above. The collected bone marrow cells were incubated with 1×RBC lysis buffer (Biolegend) for 5 minutes at room temperature to hemolyze erythrocytes. Then, the supernatant was removed by centrifugation and precipitated cells were collected. The collected cells were suspended in α-MEM medium (Invitrogen) prepared to contain 10 μM Y27632 (Tocris bioscience), 15 vol % FBS (Fetal Bovine Serum, Sigma-Aldrich), 1 vol % penicillin/streptomycin (Nacalai Tesque), 1×NEAA (non-essential amino acids for MEM, Gibco), 55 μM 2-mercaptoethanol (Gibco), 10 mM HEPES (2-[4-(2-Hydroxyethyl)-1-piperazinyl]ethanesulfonic acid, Nacalai Tesque), and 1× GlutaMAX™ Supplement (Invitrogen), and 5×10⁵ cells were seeded and cultured in each well of a collagen I-coated plate. The culture conditions were 37° C., 5% O₂, and 5% CO₂. The medium was changed every 3 days. On the 10th day of culture, the medium was removed, the wells were washed with 1×PBS (Nacalai Tesque), and colonies were stained with 0.05 wt/vol % crystal violet (Nacalai Tesque) for 30 minutes. Among the stained colonies, those considered to be mesenchymal stem cell colonies based on their characteristics such as shape, size, and cell density were counted, and P value was calculated by two-way ANOVA.

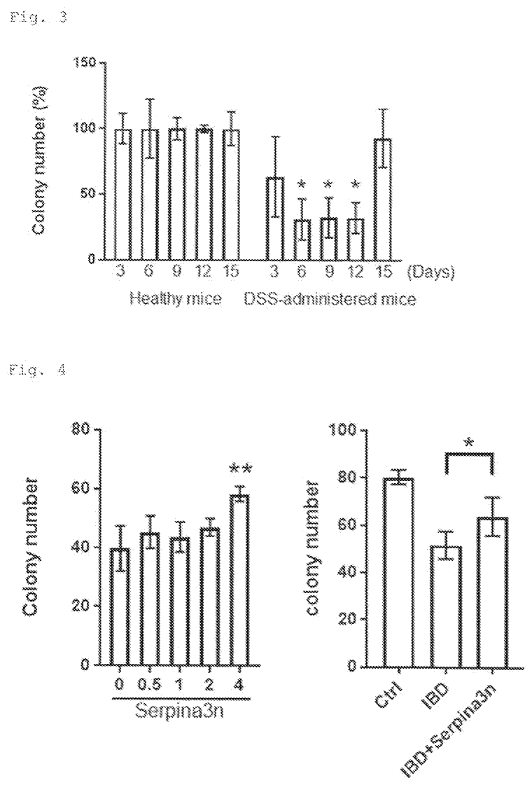

It has been well known that when bone marrow cells are collected and cultured on a solid phase, adherent cells containing mesenchymal stem cells adhere to the solid phase to proliferate. Among the mesenchymal stem cells contained in the adherent cells, mesenchymal stem cells having colony-forming property proliferate while forming colonies. The number of formed mesenchymal stem cell colonies had a tendency to decrease in the bone marrow cells collected on the 3rd day from the start of DSS administration, and in the bone marrow cells collected on the 6th, 9th, and 12th days, the number of mesenchymal stem cells colonies significantly decreased (FIG. 3). It was also observed when the same experiment was performed using the vertebrae that the number of formed mesenchymal stem cell colonies had a tendency to decrease. These results indicate that bone marrow mesenchymal stem cells are reduced in DSS-induced IBD model mice.

### 3. Increase of Colony-Forming Cells by Serpin A3N In Vitro

Bone marrow cells were collected from the femurs of DSS-administered mice on the 9th day from the start date of DSS administration and healthy mice normally bred during the same period. DSS was administered in the same manner as in section 1 above. The collected bone marrow cells were incubated with 1×RBC lysis buffer (Biolegend) for 5 minutes at room temperature to hemolyze erythrocytes. Then, the supernatant was removed by centrifugation and precipitated cells were collected. The collected cells were seeded in each well of a collagen I-coated plate at 5×10⁵ cells, and cultured in α-MEM medium (Invitrogen) prepared to contain 10 μM Y27632 (Tocris bioscience), 15 vol % FBS (Fetal Bovine Serum, Sigma-Aldrich), 1 vol % penicillin/streptomycin (Nacalai Tesque), 1×NEAA (non-essential amino acids for MEM, Gibco), 55 μM 2-mercaptoethanol (Gibco), 10 mM HEPES (2-[4-(2-Hydroxyethyl)-1-piperazinyl]ethanesulfonic acid, Nacalai Tesque), and 1× GlutaMAX™ Supplement (Invitrogen). The culture conditions were 37° C., 5% 02, and 5% CO₂. At the start of culture, mouse serpin A3N (R&D systems, catalog number: 4709-PI (4709-PI-010)) (SEQ ID NO: 28) (6-His tag added to its C-terminus) or PBS (1×PBS, Nacalai Tesque, catalog number: 14249-24) was added to the medium. Specifically, for the addition of serpin A3N, 1 μL of 500 ng/mL serpin A3N stock solution was diluted with 1.25 mL of the α-MEM medium prepared as above to provide a 400 ng/mL serpin A3N medium. The serpin A3N medium and the α-MEM medium prepared as above were mixed to provide a medium containing a predetermined final concentration of serpin A3N, and then the cells were suspended in this medium and seeded in each well. For comparison, 1 μL of PBS was used instead of 1 μL of the serpin A3N stock solution. The medium was changed every 3 days and the medium containing PBS or serpin A3N was used again at the time of medium change. On the 10th day of culture, the medium was removed, the wells were washed with 1×PBS (Nacalai Tesque), and colonies were stained with 0.05 wt/vol % crystal violet (Nacalai Tesque) for 30 minutes. Among the stained colonies, those considered to be mesenchymal stem cell colonies based on their characteristics such as shape, size, and cell density were counted, and P value was calculated by two-way ANOVA.

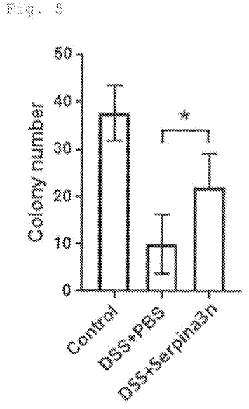

When the bone marrow cells collected from healthy mice were cultured in the presence of serpin A3N (4 ng/mL), a significant increase in the number of mesenchymal stem cell colonies was observed (FIG. 4, left). While a decrease in the number of mesenchymal stem cell colonies was observed in the bone marrow cells collected from DSS-administered mice (FIG. 3), the number of mesenchymal stem cell colonies was significantly restored by addition of serpin A3N (4 ng/mL). (FIG. 4, right).

### 4. Increase of Colony-Forming Cells by Serpin A3N In Vivo

As described in section 1 above, 1.5% DSS was administered to mice for 6 days to induce colitis. The mice were divided into two groups, and in addition to the oral administration of DSS, for 6 days from the start date of DSS administration to the 5th day, PBS (1×PBS, Nacalai Tesque, catalog number: 14249-24) (100 μL) containing serpin A3N (R&D systems, catalog number: 4709-PI) (400 ng) was administered to one group and PBS (100 μL) containing no serpin A3N was administered to another group, by intravenous injection once daily in one shot. On the 9th day from the start date of DSS administration, bone marrow cells were collected from the femurs of each group, and the collected bone marrow cells were incubated with 1×RBC lysis buffer (Biolegend) for 5 minutes at room temperature to hemolyze erythrocytes. Then, the supernatant was removed by centrifugation and precipitated cells were collected. The collected cells were seeded in each well of a collagen I-coated plate at 5×10⁵ cells, and cultured in α-MEM medium (Invitrogen) prepared to contain 10 μM Y27632 (Tocris bioscience), 15 vol % FBS (Fetal Bovine Serum, Sigma-Aldrich), 1 vol % penicillin/streptomycin (Nacalai Tesque), 1×NEAA (non-essential amino acids for MEM, Gibco), 1 vol % monothioglycerol (Wako), 10 mM HEPES (2-[4-(2-Hydroxyethyl)-1-piperazinyl]ethanesulfonic acid, Nacalai Tesque), and 1× GlutaMAX™ Supplement (Invitrogen). The culture conditions were 37° C., 5% O₂, and 5% CO₂. The medium was changed every 3 days. On the 10th day of culture, the medium was removed, the wells were washed with 1×PBS, and colonies were stained with 0.05 wt/vol % crystal violet for 30 minutes. Among the stained colonies, those considered to be mesenchymal stem cell colonies based on their characteristics such as shape, size, and cell density were counted, and P value was calculated by one-way ANOVA.

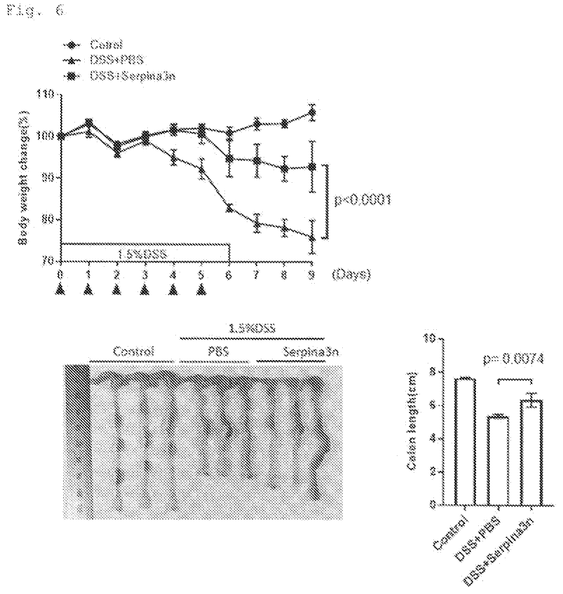

While a decrease in the number of mesenchymal stem cell colonies was observed in the bone marrow collected from DSS-administered mice (FIG. 3), administration of serpin A3N to DSS-administered mice significantly restored the number of mesenchymal stem cell colonies (FIG. 5).

The above results demonstrated that serpin A3N promoted growth or suppressed decrease of colony-forming mesenchymal stem cells in bone marrow cells.

### 5. Effects of Serpin A3N on IBD Model Mice (1)

The drinking water containing 1.5 wt/vol % DSS (MP biomedicals, catalog number: 160110) was administered to 8-week-old C57BL/6J male mice (6 animals) for 6 days to induce colitis. From the 6th day, normal drinking water containing no DSS was administered. The mice were divided into two groups, and in addition to the oral administration of DSS, for 6 days from the start date of DSS administration to the 5th day, PBS (1×PBS, Nacalai Tesque, catalog number: 14249-24) (100 μL) containing mouse serpin A3N (R&D systems, catalog number: 4709-PI-010) (400 ng) was administered to one group and PBS (100 μL) containing no serpin A3N was administered to another group, by intravenous injection once daily in one shot. The body weight change in each mouse was measured daily. The large intestine was collected from each mouse on the 9th day from the start date of DSS administration, and a large intestine image was taken with a camera. Then, the colon length was calculated from image thus obtained using ImageJ software. The p-value was calculated by two-way ANOVA.

The weight loss in DSS-administered mice was significantly suppressed by administration of serpin A3N (FIG. 6, top). Also, the decrease in the colon length in DSS-administered mice was also significantly suppressed by administration of serpin A3N (FIG. 6, bottom). These results demonstrate that serpin A3N can treat IBD.

### 6. Effects of Serpin A3N on IBD Model Mice (2)

The drinking water containing 1.5 wt/vol % DSS (MP biomedicals, catalog number: 160110) was administered to 8-week-old C57BL/6J male mice (6 animals) for 6 days to induce colitis. From the 6th day to the 20th day, the drinking water was changed to normal drinking water containing no DSS. Then, for 6 days from the 21st day, the drinking water containing DSS as above was administered again. Subsequently, from the 27th day, normal drinking water was administered. The mice were divided into two groups, and in addition to the oral administration of DSS, for 6 days from the first start date of DSS administration (the start date of the experiment, day 0) to the 5th day, PBS (1×PBS, Nacalai Tesque, catalog number: 14249-24) (100 μL) containing mouse serpin A3N (R&D systems, catalog number: 4709-PI-010) (400 ng) was administered to one group and PBS (100 μL) containing no serpin A3N was administered to another group, by intravenous injection once daily in one shot.

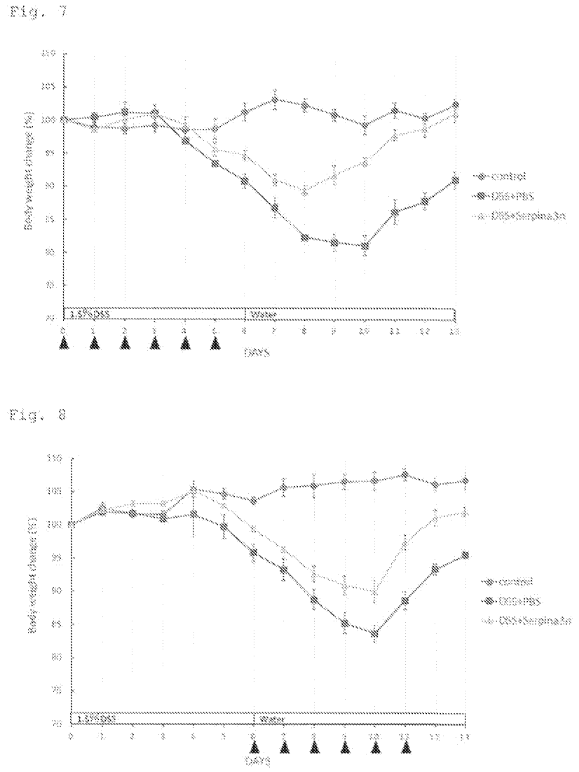

FIG. 7 shows the measurement results of body weight change in the mice from the start date of the experiment to the 13th day, together with the measurement results of the normally bred healthy mice (DSS non-administered, control). FIG. 7 shows the body weight change of the mice with the body weight on the start date of the experiment as 100%. The weight loss in DSS-administered mice was significantly suppressed by administration of serpin A3N, and weight recovery after discontinuation of DSS administration was also better in the serpin A3N-administered group (FIG. 7). In addition, from the discontinuation of DSS administration on the 6th day to the 30th day, the body weight of the mice in the serpin A3N-administered group was higher than that in the non-administered group.

### 7. Effects of Serpin A3N on IBD Model Mice (3)

The drinking water containing 1.5 wt/vol % DSS (MP biomedicals, catalog number: 160110) was administered to 8-week-old C57BL/6J male mice (6 animals) for 6 days to induce colitis. From the 6th day to the 20th day, the drinking water was changed to normal drinking water containing no DSS. Then, for 6 days from the 21st day, the drinking water containing DSS as above was administered again. Subsequently, from the 27th day, normal drinking water was administered. The mice were divided into two groups, and for 6 days from the 6th day, at which the first oral DSS administration was discontinued, to the 11th day, PBS (1×PBS, Nacalai Tesque, catalog number: 14249-24) (100 μL) containing mouse serpin A3N (R&D systems, catalog number: 4709-PI-010) (1 μg) was administered to one group and PBS (100 μL) containing no serpin A3N was administered to another group, by intravenous injection once daily in one shot.

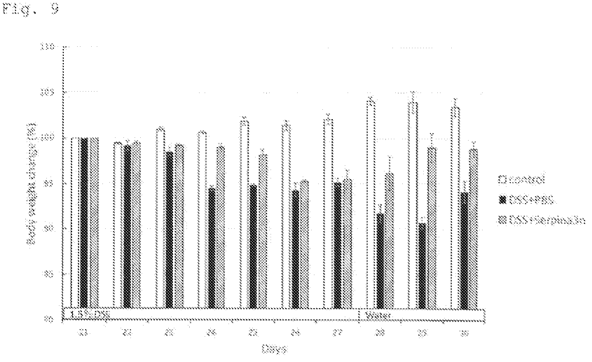

FIG. 8 shows the measurement results of body weight change in the mice from the start date of the first DSS administration (start date of the experiment, day 0) to the 13th day, together with the measurement results of the normally bred healthy mice (DSS non-administered, control). FIG. 8 shows the body weight change in the mice with the body weight on the start date of the experiment as 100%. The weight loss in DSS-administered mice was significantly suppressed by administration of serpin A3N even after DSS administration, and weight recovery after discontinuation of DSS administration was also better in the serpin A3N-administered group (FIG. 8).

### 8. Effects of Serpin A3N on IBD Model Mice (4)

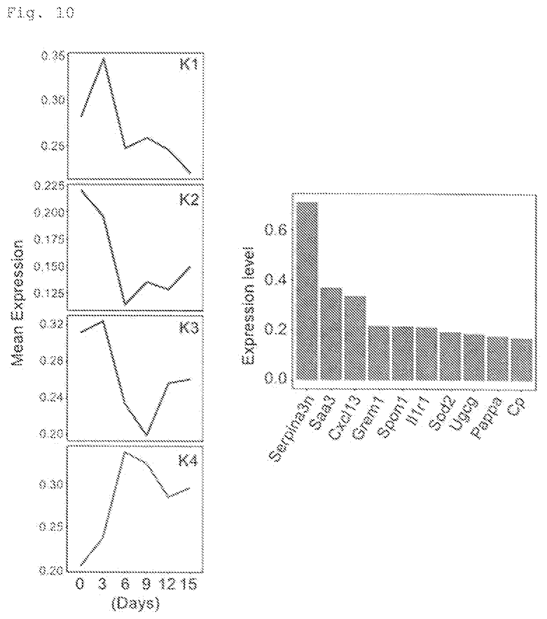

In the experiment of section 6 above, effects of serpin A3N on the colitis induced in the mice by additional DSS administration were evaluated. FIG. 9 shows the body weight change in each mice from the 21st day from the start date of the experiment (the day on which the additional DSS administration was started) based on the body weight of the 21st day (100%). For comparison, the measurement results of body weight of normally bred healthy mice (DSS non-administered, control) are shown together.

The body weight of DSS non-administered healthy mice (control) increased steadily, while the body weight of DDS-administered mice (DSS+PBS, DSS+Seripina3n) decreased. The weight loss in the group receiving serpin A3N at the time of initial induction with DSS (DSS+Seripina3n) was clearly suppressed as compared with the group not receiving serpin A3N (DSS+PBS) (FIG. 9). That is, serpin A3N was shown to have the effects of maintaining remission and suppressing relapse of IBD.

### 9. Expression of Serpin A3N in IBD Model Mice (Single-Cell RNA-Sequencing Analysis)

The drinking water containing 1.5 wt/vol % DSS (MP biomedicals, catalog number: 160110) was administered to 8-week-old C57BL/6J male mice (18 animals) for 6 days to induce colitis. From the 6th day, normal drinking water containing no DSS was administered. On the 0th, 3rd, 6th, 9th, 12th, and 15th days from the start date of DSS administration, the large intestine was collected from 3 mice each. Then, cells of the collected large intestine were dissociated and dispersed to prepare a cell suspension. By using FACS (device name: BD FACSAria™III, Becton, Dickinson and Company), dead cells in the cell dispersion were removed and the whole living cells were isolated (single cell preparation). A total of 14624 cells were obtained from 18 mice and 6 different time points. Then, a sequencing library was constructed according to the smart-seq2 method. The library thus prepared was sequenced with a sequencer (device name: NextSeq 500, Illumina, Inc). That is, profiles were obtained for a total of 14624 cells from 18 mice and 6 different time points.

Clustering according to the UMAP method was performed for all the obtained sequence data. As a result, the cells of the large intestine of the IBD model mouse were clustered into 15 clusters. The cell type of each cluster was identified based on the labeled gene. For example, the cell type of one cluster was identified as a stromal cell by three labeled genes (Collal, Pdgfra, Spon2).

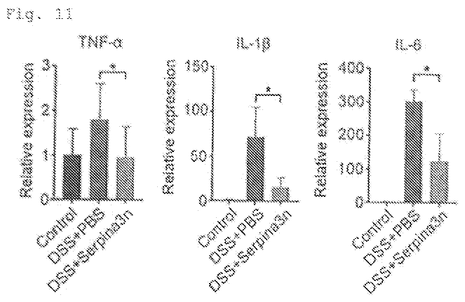

Also, to detect differential gene expression at various stages of inflammation (days after induction), expression genes in stromal cells of the large intestine were analyzed for each sampling day, and 257 differentially expressed genes (DEGs), genes whose expression levels were significantly differentiated, were identified. Analyzing these differentially expressed genes based on K-means clustering enabled grouping of genes with a similar expression pattern, and the 257 differentially expressed genes were classified into four subclusters (K1 to K4) (FIG. 10, left graph). One of these subclusters K4 was a cluster to which genes whose expression peaked on the 6th to 9th days from the start date of DSS administration belonged, and it was demonstrated that serpin A3N was the gene having the highest expression level in this subcluster K4 (FIG. 10, right graph).

### 10. Effects of Serpin A3N on IBD Model Mice (4)

The drinking water containing 1.5 wt/vol % DSS (MP biomedicals, catalog number: 160110) was administered to 8-week-old C57BL/6J male mice (6 animals) for 6 days to induce colitis. From the 6th day to the end of the experiment (9th day), normal drinking water containing no DSS was administered. The mice were divided into two groups, and in addition to the administration of DSS, for 6 days from the start date of DSS administration to the 5th day, PBS (1×PBS, Nacalai Tesque, catalog number: 14249-24) (100 μL) containing mouse serpin A3N (R&D systems, catalog number: 4709-PI-010) (400 ng) was administered to one group and PBS (100 μL) containing no serpin A3N was administered to another group, by intravenous injection once daily in one shot. The large intestine was collected from the mice on the 9th day from the start date of DSS administration.

From the collected mouse large intestine, total RNA was extracted with ISOGENE (Nippon Gene Co., Ltd.) according to the manufacturer's protocol, and further purified with RNeasy Plus Mini kit (QIAGEN). The concentration of total RNA in the sample solution after purification was measured with a fluorometer (Qubit 3.0 Fluorometer, Thermo Fisher Scientific). Then, the amount of RNA was adjusted appropriately (to 500 ng) based on the measured concentration to prepare a sample for quantitative RT-PCR (quantitative reverse transcription-polymerase chain reaction, RT-qPCR). With this sample, the expression levels of TNF-α, IL-1β, and IL-6 in the large intestine were measured by quantitative RT-PCR. Specifically, with a cDNA synthesis kit (iScript reverse transcription supermix for RT-qPCR, Bio-Rad Laboratories), cDNA was synthesized from the total RNA according to the instruction manual. Then, quantitative RT-PCR was carried out with the synthesized cDNA, a mixed reagent (THUNDERBIRD SYBR qPCR mix, TOYOBO), and a predetermined primer set.

The primer sets used were shown below.

TNF-α
(forward)
SEQ ID NO: 32
5′-GCTCCAGTGAATTCGGAAAG-3′
(reverse)
SEQ ID NO: 33
5′-GATTATGGCTCAGGGTCCAA-3′
IL-1β
(forward)
SEQ ID NO: 34
5′-TGAGCACCTTCTTTTCCTTCA-3′
(reverse)
SEQ ID NO: 35
5′-TTGTCTAATGGGAACGTCACAC-3′
IL-6
(forward)
SEQ ID NO: 36
5′-TCTAATTCATATCTTCAACCAAGAGG-3′
(reverse)
SEQ ID NO: 37
5′-TGGTCCTTAGCCACTCCTTC-3′
actb
(forward)
SEQ ID NO: 38
5′-CTAAGGCCAACCGTGAAAAG-3′
(reverse)
SEQ ID NO: 39
5′-ACCAGAGGCATACAGGGACA-3′

The measurement was repeated 3 times. Then, with the CFX manager software (Bio Rad Laboratories), based on the standard curve method, the expression levels of the above genes (TNF-α, IL-1β, IL-6) in the serpin A3N-administered group (DSS+Serpina3n) and the non-administered group (DSS+PBS) were compared with the expression levels of these genes in the normally bred healthy mice (DSS non-administered, control), which were determined to be 1. The expression levels were corrected by using β-actin (actb) as an internal standard gene. The results are shown in FIG. 11.

As can be seen from FIG. 11, it was observed that DSS administration increased the expression levels of TNF-α, IL-1β, and IL-6 genes, but administration of serpin A3N suppressed these expressions. That is, it was suggested that administration of serpin A3N suppressed production of inflammatory cytokines TNF-α, IL-1β, and IL-6.

1-12. (canceled)

13. A method for promoting growth or suppressing decrease of mesenchymal stem cells, comprising administering serpin A3 to a subject in need thereof.

14. The method according to claim 13, wherein the mesenchymal stem cells are colony-forming mesenchymal stem cells.

15. The method according to claim 13, wherein the mesenchymal stem cells are bone marrow mesenchymal stem cells.

16. The method according to claim 13, wherein the subject suffers from inflammatory bowel disease.

17. The method according to claim 13, wherein the decrease of mesenchymal stem cells is caused by inflammatory bowel disease.

18. The method according to claim 13, wherein the serpin A3 is selected from the group consisting of:
a) a polypeptide comprising or consisting of an amino acid sequence of a full-length or mature serpin A3,
b) a polypeptide comprising an amino acid sequence of a mature serpin A3 and consisting of a partial amino acid sequence of a full-length serpin A3,
c) a polypeptide comprising or consisting of a partial amino acid sequence of a full-length or mature serpin A3 and having an activity of promoting growth or suppressing decrease of mesenchymal stem cells,
d) a polypeptide comprising or consisting of an amino acid sequence that differs from an amino acid sequence of a full-length or mature serpin A3 in that 1 to 10 amino acids are substituted, deleted, inserted or added, and having an activity of promoting growth or suppressing decrease of mesenchymal stem cells,
e) a polypeptide comprising or consisting of an amino acid sequence having about 90% or more sequence identity with an amino acid sequence of a full-length or mature serpin A3 and having an activity of promoting growth or suppressing decrease of mesenchymal stem cells,
f) a polypeptide encoded by a DNA that hybridizes to a nucleic acid sequence encoding a full-length or mature serpin A3 under a stringent condition and having an activity of promoting growth or suppressing decrease of mesenchymal stem cells, and
g) a polypeptide encoded by a nucleic acid sequence having about 90% or more sequence identity with a nucleic acid sequence encoding a full-length or mature serpin A3 and having an activity of promoting growth or suppressing decrease of mesenchymal stem cells.

19. The method according to claim 18, wherein the serpin A3 is the polypeptide of a) or b).

20. The method according to claim 18, wherein the full-length serpin A3 comprises the amino acid sequence of SEQ ID NO: 1, 11, 12, or 27.

21. The method according to claim 18, wherein the mature serpin A3 comprises the amino acid sequence of SEQ ID NO: 24, 25, 26 or 28.

22. The method according to claim 18, wherein the nucleic acid sequence encoding a full-length serpin A3 comprises the nucleic acid sequence of SEQ ID NO: 29, 30, or 31.

23. A method for treating inflammatory bowel disease, comprising administering serpin A3 to a subject in need thereof.

24. The method according to claim 23, wherein the serpin A3 is selected from the group consisting of:
a) a polypeptide comprising or consisting of an amino acid sequence of a full-length or mature serpin A3,
b) a polypeptide comprising an amino acid sequence of a mature serpin A3 and consisting of a partial amino acid sequence of a full-length serpin A3,
c) a polypeptide comprising or consisting of a partial amino acid sequence of a full-length or mature serpin A3 and having an activity of promoting growth or suppressing decrease of mesenchymal stem cells,
d) a polypeptide comprising or consisting of an amino acid sequence that differs from an amino acid sequence of a full-length or mature serpin A3 in that 1 to 10 amino acids are substituted, deleted, inserted or added, and having an activity of promoting growth or suppressing decrease of mesenchymal stem cells,
e) a polypeptide comprising or consisting of an amino acid sequence having about 90% or more sequence identity with an amino acid sequence of a full-length or mature serpin A3 and having an activity of promoting growth or suppressing decrease of mesenchymal stem cells,
f) a polypeptide encoded by a DNA that hybridizes to a nucleic acid sequence encoding a full-length or mature serpin A3 under a stringent condition and having an activity of promoting growth or suppressing decrease of mesenchymal stem cells, and
g) a polypeptide encoded by a nucleic acid sequence having about 90% or more sequence identity with a nucleic acid sequence encoding a full-length or mature serpin A3 and having an activity of promoting growth or suppressing decrease of mesenchymal stem cells.

25. The method according to claim 24, wherein the serpin A3 is the polypeptide of a) or b).

26. The method according to claim 24, wherein the full-length serpin A3 comprises the amino acid sequence of SEQ ID NO: 1, 11, 12, or 27.

27. The method according to claim 24, wherein the mature serpin A3 comprises the amino acid sequence of SEQ ID NO: 24, 25, 26 or 28.

28. The method according to claim 24, wherein the nucleic acid sequence encoding a full-length serpin A3 comprises the nucleic acid sequence of SEQ ID NO: 29, 30, or 31.

29. A method for promoting growth or suppressing decrease of mesenchymal stem cells, comprising culturing mesenchymal stem cells in a medium containing serpin A3.


SEQUENCE LISTING (39 sequences)

SEQ ID NO: 1 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 2 - protein, 422 aa, Mus musculus
MAFIAALGLLMAGICPAITYWATADGQLGRHTEVQKDRDHEIQLDSVTLASINTDFAFSLYKKLALKNPHKNIVFSPLSISAALALMSLGAKDNTLEEILEGLKFNLTETPEADIHQNFGHLLQMLIQPENQVQINAGNALFIDKHLQILTEFKEKARALYKAEAFTADFQLPREATKLINDYVRKQTQGKIKELVSDLHRNTSMALVNFLNFQGFWNVTFDPEDTFLGNFTLDRKRTVNVPMMKTEELTTNYFRDEEMQSTVMELNYIGNASFLFILPDQGRIQHVEDSLQPQSLRKWRKSLRPRMLDELSLPKFSLSQDYNLNDILPELGIKEVFSTQADLSGITGAKNIRVSQMIHQAALDVTETHTEADVITIARYNFQSAKIKAKIVKVDREFLYLILDPMFKSISVMGKVINPLTN

SEQ ID NO: 3 - protein, 420 aa, Mus musculus
MAFIAALGLLMAEICPAVICCSDGTLGMHNAVQKGQDTQKQLDSLTLASINTDFAFSFYKELALKNPHKNIAFSPFGIATALNSLTLGAKGNTLEEILEVLKFNLTETSEADIHQGFKHLLQRLSHPGDQVQIRTGNALFVEKHLQILAEFKEKARALYHTEVFTANFQQPHEAMKLINSYMSNQTQGKIKELVSDMDGNTSMVIVNDLFFKAEWMVPFNSDDTFMGKFIVDRSRHVKVPMMKTKNLRTPYFRDEELKCTVVELNYKGNGKAMFILPDQGKMQQVEASLQPGTLKKWRKSLRPRKIKELHLPKFSLSQHYNLEDILPELGIRELFSTQADLSGITGVKNITVSEMIHSTELDMTEKGTEGDAITIVGYNFMSAKLKPVFVKFEDQFLYIVLDQGDLWIHVMGKVINPSEK

SEQ ID NO: 4 - protein, 417 aa, Mus musculus
MAFIVALGLVITGICPGVLCFPDGTLERDTLFHKDKENGTQLDSLTLASINTDFAFSLYKKLALKNPDTNIVFSPLSISAALAIVSLGAKGNTLEEILEGLNFNLTETPEADIHQGFGHLLQRLSHPGEQVQISTGSALFVEKHLQILAEFQEKARALYQAEAFTADFQQPLEATKLINDYVSNQTQRKIKGLISDLDTDTLMVLVNYIYFKGKWKMPFNPRDTFESEFYLDVKRSVKVPMMKIKTLTTPYFRDEELSCTVVELKYKGNASALFILPDQGRMQQVEASLQPETLRKWKNSLRPRKMGELYLPKFSISTDYSLKNILPELGIKEIFSKQADLSGITGTKDLIVSQMVHKAVLDVAETGTEGVAATGVNFRILSRRTSLWFNRTFLMVISHTDVQTTLFIAKITHPKRA

SEQ ID NO: 5 - protein, 445 aa, Mus musculus
MAGVSPAVFGCPDVTLGRNTAVREVQENITSVDSLTLASSNTDFAFSLYKELVLKNPDENVVFSPFSICTALALLSLGAKSNTLKEILEGLKFNLTETPEPDIHQGFRYLLDLLSQPGNQVQISTGSALFIEKHLQILAEFKEKARALYQAEAFTADFQQPLEATKLINDYVSNHTQGKIKELISDLDKRTLMVLVNYIYFKGKWEMPFDPDDTCKSEFYLDENRSVKVPMMKINNLTTPYFRDEELSCTVVELKYTGNASAMFILPDQGKMQQVEASLQPETLRNWKDSLKPRLINELCLPKFSISTDYSLEHILPELGIRELFSTQADLSAITGTKDLRTSQVVHKAVLDVAETGTEAAAGTGYQNLQCCQGVIYSMKIYFDRPFLMIISDTNTHIALFMAKVSNPESDENFLNVEYAFPQVLEIMPEYRSVCTCCLPCLTRQ

SEQ ID NO: 6 - protein, 440 aa, Mus musculus
MAGVSPAVFGCPDVTLGRNTAVREVQENVTSVDSLTLVSSNTDFAFSLYRKLVLKNPDENVVFSPFSICTALALLSLGAKSNTLKEILEGLKFNLTETPEPDIHQGFRYLLDLLSQPGNQVQISTGSALFIEKHLQILAEFKEKARALYQAEAFTADFQQPLKATKLINDYVSNHTQGKIKQLISGLKESMLMVLVNYIYFKGKWKNPFDPNDTFKSEFYLDEKRSVIVSMMKTGYLTTPYFRDEELSCTVVELKYTGNASAMFILPDQGRMQQVEASLQPETLRKWKNSLKPRMIHELRLPKFSISTDYSLEHILPELGIREVFSTQADLSAITGTKDLRVSQVVHKAVLDVAEKGTEAAAATGMAGVGCCAVFDFLEIFFNRPFLMIISDTKAHIALFMAKVTNPERSENFPNGEGASSQRLESKRLCFGDPLCLIGQ

SEQ ID NO: 7 - protein, 408 aa, Mus musculus
MAGVSPAVLGCPDVTLERNTAVHEVQENITSGDSLTVASSNTDFAFSLYRKLVLKNPDENVVFSPFSIFTALALLSLGAKSNTLKEILEGLKFNLTETPEPDIHQGFRYLLDLLSQPGDQVQISTGSALFVEKHLQILAEFKEKARALYQAEAFTADFLQPCQAKKLINDYVSNQTQGKIKELISDLDKSTLMVLVNYIYFKGKWKMPFDPRDTFNSKFYLDEKRSVKVPMMKIEELTTPYFRDDELSCSVVELKYTGNASALFILPDQGKMQQVETSLHPETLRKWKNSLKPSRISELHLPKFSISNDYSLEHVLPVLGIREVFSMQADLSAITGTMDLRVSQVVHKAVLDVTETGTEAAAATGVKVNLRCGKIYSMTIYFKRPFLIIISDINTHIALFMAKVTNPK

SEQ ID NO: 8 - protein, 420 aa, Mus musculus
MAFIAALGLLMAGICPAVLCCPEDTLGKHTPVQKDRDHETQLDSLTLASINTDFAFSLYKKLALKNPHKNFVFSPLSITIALASLSLGAKGNTLEEILEGLKFNLTETPEADIHQGFGHLLQRLSQPGDQVQISTGNSMVVEKHLQILAEFKEKARALYHTEVFTADFQQPREARKLLNDYVSNQTQGMIKELVSDLEERTSMVMTNFALFNGKWNMTFDPYETFMGTFIEDRRTPVKVSMMKMKELRAPYFRDEKMKCTVVELNYKGNGKAMFILPDQGKMKQVEASLQPATLRGWRKSLRPRMIDELYLPKFSISKNYRLENILPELGIKEVFSTQADLSGISGGKDVRVSRMFHSAALDMTETGTEARATTRDKYDFLSTKSNPTVVNLNTPFLFCVLHSDSENIDFMGKINNPAQN

SEQ ID NO: 9 - protein, 418 aa, Mus musculus
MAFIVAMGMILMAGICPAVLCFPDGTKEMDIVFHEHQDNGTQDDSLTLASVNTDFAFSLYKKLALKNQDKNIVFSPLSISAALALVSLGAKGKTMEEILEGLKFNLTETPEADIHQGFGNLLQSLSQPEDQDQINIGNAMFIEKDLQILAEFHEKTRALYQTEAFTADFQQPTEAKNLINDYVSNQTQGMIKKLISELDDGTLMVLVNYIYFKGKWKISFDPQDTFESEFYLDEKRSVKVPMMKMKLLTARHFRDEELSCSVLELKYTGNASALLILPDQGRMQQVEASLQPETLRKWRKTLFSSQIEELNLPKFSIASDYRLEEDVLPEMGIKEVFTEQADLSGITEAKKLSVSQVVHKAVLDVAETGTEAAAATGVIGGIRKAVLPAVCFNRPFLIVIYHTSAQSILFMAKVNNPK

SEQ ID NO: 10 - protein, 418 aa, Mus musculus
MAFIAALGILMAGICPTVLCFSDDTWGIDILLHKNQESGTPDDSLTLASINTDFAFSLYKKMALKNPDKNIVFSPLSISAALALVSLGAKGNTLEEILEGLKFNLTETSEADIHQGFGHLLQRLSQPEDQDQINIGNAMFIEKDLQILAEFHEKTRALYQTEAFTADFQQPTEATKLINDYVSNQTQGMIKKLISELDDRTLMVLVNYIYFKGKWKISFDPQDTFESEFYLDEKRSVKVPMMKMKFLTTRHFRDEELSCSVLELKYTGNASALFILPDQGRMQQVEASLQPETLRKWWKSLKTRKIGELYLPKFSISTDYNLKDILPELGIKEIFSKQADLSGITGTKDLSVSQVVHKAVLDVAETGTEAAAATGFIFGFRSRRLQTMTVQFNRPFLMVISHTGVQTTLFMAKVTNPK

SEQ ID NO: 11 - protein, 418 aa, Mus musculus
MAFIAALGLLMAGICPAVLCFPDGTLGMDAAVQEDHDNGTQLDSLTLASINTDFAFSLYKELVLKNPDKNIVFSPLSISAALAVMSLGAKGNTLEEILEGLKFNLTETSEADIHQGFGHLLQRLNQPKDQVQISTGSALFIEKRQQILTEFQEKARALYQAEAFTADFQQPRQAKKLINDYVRKQTQGMIKELVSDLDKRTLMVLVNYIYFKAKWKVPFDPLDTFKSEFYAGKRRPVIVPMMSMEDLTTPYFRDEELFCTVVELKYTGNASAMFILPDQGKMQQVEASLQPETLRKWKNSLKPRMIDELHLPKFSISTDYSLEDVLSKLGIREVFSTQADLSAITGTKDLRVSQVVHKAVLDVAETGTEAAAATGVKFVPMSAKLYPLTVYFNRPFLIMIFDTETEIAPFIAKIANPK

SEQ ID NO: 12 - protein, 408 aa, Rattus norvegicus
MDGIGSALLSFPDCILGEDTLFHEDQDKGTQLDSLTLASINTDFAFSLYKKLALRNPHKNVVFSPLSISAALAVVSLGAKGSSMEEILEGLKFNLTETPETEIHRGFGHLLQRLSQPRDEIQISTGNALFIEKRLQVLAEFQEKAKALYQAEAFTADFQQSREAKKLINDYVSKQTQGKIQGLITNLAKKTSMVLVNYIYFKGKWKVPFDPRDTFQSEFYSGKRRSVKVPMMKLEDLTTPYVRDEELNCTVVELKYTGNASALFILPDQGKMQQVEASLQPETLRRWKDSLRPSMIDELYLPKFSISADYNLEDVLPELGIKEVFSTQADLSGITGDKDLMVFQVVHKAVLDVAETGTEAAAATGVKFVPMSAKLDPLIIAFDRPFLMIISDTETAIAPFLAKIFNPK

SEQ ID NO: 13 - protein, 419 aa, Rattus norvegicus
MAFIAALGLLMAGICPAVLGFPDGTLGNDTLLHKDQDKGTQLDSLTLESINTDFAFSLYKMLALKNPDKNVVFSPLSISAALAIVSLGAKGNTLEEILEVLRFNLTESYETDIHQGFGHLLQRLSQPGDQVKIITGNALFIDKNLQVLAEFQEKTRALYQVEAFTADFQQPRVTEKLINDYVRNQTQGKIQELVSGLKERTSMVLVNYLLFRGKWKVPFDPDYTFESEFYVDEKRSVKVSMMKIEELTTPYFRDEELSCSVLELKYTGNSSALFILPDKGRMQQVEASLQPETLKKWKDSLRPRKIDELYLPRLSISTDYSLEEVLPELGIRDVFSQQADLSRITGAKDLSVSQVVHKVVLDVNETGTEAAAATGANLVPRSGRPPMIVWFNRPFLIAVSHTHGQTILFMAKVINPVGA

SEQ ID NO: 14 - protein, 424 aa, Macaca mulatta
MERMLPLLALGLLVAGFCPAVLCYPNCPLDKENPTQEDQDRGTHVDLGLASANVDFAFSLYKQLVLKAPDKNVIFSPLSISTALAFLSLGAHNTTLMEILRGLKFNLTETSEAEIHQSFQHLLRTLNQSSDGLQLSMGNAMFIEEQLSLLDRFMEDAKRLYGSEAFATDFQDSAVAKKFINDYVKNRTRGKITDLIEDLDSQTVMVLVNYIFFKAKWKMPFDPHDTHDSRFYWSKRRWVKVPMMSLQHVTTPYFRDEELSCTVVELKYIGNASALFILPDQDKMEEVEAMLLPETLKRWKDSLEFRHIDELYLPKFSLSRDYDLEDVLRQLGIEEVFTSEADLSGITGARNLAVSQVVVHKAVLDVSEEGTEASAATGVKITLLSAFVDPKITVRFNRPFLMIIVPMDTQNIFFISKVINPKQA

SEQ ID NO: 15 - protein, 423 aa, Pongo abelii
MERMLPFLALGLLVAGFCPAVLCHPNCPLDEENPTQENQDRGTHVDLGLASTNVDFAFSLYKQLVLKAPDKNVIFSPLSISTALAFLSLGAHNTTLTEILTGLRFNLTETSEAEIHQSFQHLLRTLNQSSDELQLSMGNAMFVEEQLSLLDRFMEDAKRLYGSEAFATDFQDSAAAKKLINDYVKNRTRGKITDLIKDLDSQTMMVLVNYIFFKAKWKMPFDPQDTHQSRFYLSKKKWVMVPMMSLHHLTTPYFRDEELSCTVVELKYTGNASALFILPDQDKMEEVEAMLLPETLKRWRDSLEFRRIDELYLPKFSISRAFNLENILLQLGIVEAFTSKADLSGITGARNLVVSQVVHKAVLDVFEEGTEASAATAVKITLLSALVDPMTIVRFNRPFLMIIVPTDTQNLLFISKVINPKQA

SEQ ID NO: 16 - protein, 411 aa, Bos taurus
MRAERTSFLLALGLLMAGIRSVHCLPENVVVKDQRRRVDSHTLASSNTDFAFSLYKQLALKNPNKNVMFSPLSVSMALAFLSLGARGPTLTEILEGLKFNLTEIQETQIHQGFQHLLQALNRPSNQLQLSVGNAMFVQEELKLLDKFIEDARVLYSSEAFPTNFRDSEAARSLINDYVKNKTQGKIEELFKYLSPRTVLVLVNYIYFKAQWKTRFDPKHTEQAEFHVSKNKTVEVPMMTLDLETPYFRDKELGCMLVELTYSSNDSALFILPDEGKMQDLEAKLTPETLTRWRNSLQPRRIHELYLPKFSIKSNYELNDTLSQMGIKKIFTDADLSGITGTADLVVSQVVHGAALDVDEEGTEGAAATGIGIERTFLRIIVRVNRPFLIAVVLKDTQSIIFLGKVTNPSEA

SEQ ID NO: 17 - protein, 411 aa, Bos taurus
MRAERTSFLLALGLLVAGIPSVHCLPENVVVKDQHRRVDGHTLASSNTDFAFSLYKQLPLKNPNKNVILSPLSVSIALAFLSLGARGSTLTEILEGLKFNLTEIQEKEIHHSFQHLLQALNQPSNQLQLSVGNAMFVQEELKLLDKFIEDAQVLYSSEAFPTNFRDSEAARSLINDYVKNKTQGKIEELFKYLSPRTELVLVNYIYFKAQWKTPFDPKHTEQAEFHVSDNKTVEVPMMTLDLETPYFRDEELGCTLVELTYTSNDSALFILPDEGKMRDLEAKLTPETLTRWRNSLQPRRIHELYLPKFSIKSNYELNDILSQLGIRKIFANADLSGITGTADLVVSQVVHGAALDVDEEGTEGAAATGISMERTILRIIVRVNRPFLIAIVLKDTQSIIFLGKVTNPSEA

SEQ ID NO: 18 - protein, 411 aa, Bos taurus
MRAERTSFLLALGLLVAGIRSVHCLPENVVVKDQHRRVDGHTLASSNTDFAFSLYKQLALKNPNKNVILSPLSVSIALAFLSLGARGSTLTEILEGLKFNLTEIQEKEIHHSFQHLLQALNQPSNQLQLSVGNAMFVQEELKLLDKFIEDAQVLYSSEAFPTNFRDSEAARSLINDYVKNKTQGKIEELFKYLSPRTELVLVNYIYFKAQWKTPFDPKHTEQAEFHVSDNKTVEVPMMTLDLETPYFRDEELGCTLVELTYTSNDSALFILPDEGKMRDLEAKLTPETLTRWRNSLQPRRIHELYLPKFSIKSNYELNDILSQLGIRKIFANADLSGITGTADLVVSQVVHGAALDVDEEGTEGVAATGIGIERTFLRIIVRVNRPFLIAVVLKDTQSIIFLGKVTNPSEA

SEQ ID NO: 19 - protein, 411 aa, Bos taurus
MRAERLSPLLALGLLVAGIRSVHCLPENVVVKDRHRRVDGHTLASSNTDFAFSLYKQLALKNPNKNVMFSPLSVSMALAFLSLGARGPTLTEILEGLKFNLTEIQETQIHQGFQHLLQALNRPRNQLQLSVGNAMFVQEELKLLDKFIEDARVLYSSEAFPTNFRDPEAAKSLINDYVKNKTQGKIEELFKDLSPRTELVLVNYVYFKAQWKTRFDPKHTEQAEFHVSDNKTVEVPMMTLDLETPYFRDEELGCTLVELTYTSNDSALFILPDKGKMQDLEAKLTPEMLTRWRNSLQPRRIHELYLPKFSIKSNYELNDTLSQMGIKKIFTDADLSGITGTADLVVSQVVHGAALDVDEEGTEGAAATGIGIERTFLRIIVRVNRPFLIAVVLKDTQSIIFLGKVTNPSEA

SEQ ID NO: 20 - protein, 414 aa, Bos taurus
MRTERVSPLLALGILVAGLCSRVHCLPENVTPEEQHKVTSVDGHSLASSNTDFAFSLYKQLALKDPNKNVIFSPLSVSIALAFLSLGAHGPTVTEILEGLKFNLTETPETEIHQGFQHLLQTFNQPSNQLQLSVGNAIFVQEELKLLDKFIEDARVLYSSEAFPTNFRDPEAAKSLINDYVKNKTQGKIEELFKDLSPRTELVLVNYVYFKAQWKTRFDPKHTEKTEFHVSDNKTVEVPMMTLDLETPYFRDEELGCTLVELTYTSNDSALFILPDKGKMQDLEAKLTPEMLTRWRNSLQPRRIHELYLPKFSIKSNYELNDTLSQMGIKKIFTDADLSGITGTADLVVSQVVHGAALDVDEEGTEGAAATGIGIERTFLRIIVRVNRPFLIAVVLKDTQSIIFLGKVTNPSEA

SEQ ID NO: 21 - protein, 417 aa, Bos taurus
MRTERTSFLLALGLLVSGFCSRVHCLPENVTPEEQHKGTSVDGHSLASSNTDFAFSLYKQLALKDPNKNVIFSPLSISIALAFLSLGAHDHTVTEILEGLKFNLTETPETEIHQGFQHLLQTFNQPSNQLQLSVGNAMFVSEELKLLDKFRKDAEAFYASEVLSTNFKDSEADVKLINEYVKNKTHGKIEKLFNDLDVLTNLILLNYIFFKAQWKTPFNPNHTYESEFHVSQNERVIVPMMTLYLETPYFRDEELGCTLVELTHTSNDSALFILPDEGKMQDLEAKLTPETLTRWRNSLQPRLIHRLRLPRFSISSHYQLKDILSQLGIKKIFTSDADFSGITDDHKLAVSHVIHKAVLDVGEEGTEGAAVTAVVMATSSLLHTLTVSFNRPFLLSIFCKETQSIIFLGKVTNPKEA

SEQ ID NO: 22 - protein, 418 aa, Bos taurus
MRAERMSPLLALGLLVSGLCSRVHCLPENVAPEERHKGTSVDGHSLASSNTDFAFSLYKQLALKNPNKNVIFSPLSISIALAFLSLGARGPTVTEILEGLKFNLTETPEREIHQGFQHLLQMLSRPSNELQLSVGNTMFVQEQLKLLDKFREDALALYTSEAFSTNFKDPETAKSLINDYVKNKTRGKIVDLFKDLDPLTKVILVNYIYFKAQWRTPFDPKQTYKSQFHVSKNKTVEVPMMSIGDLVTPYFRDEELDCTLVELTYTSNDSALFILPDEGKMQDLEAKLIPEMLTRWRESLYPRGIHELNLPRFSIATDYKLKDILSQLEIKKVFTQEADLSGITDDHELEVSQVVHKAVLDVGEEGTEGAAATGVKVGITSINNHIPLSFNRPFLIAIVLKDTQSIIFLGKVTNPSQA

SEQ ID NO: 23 - protein, 415 aa, Sus scrofa
MSLFLALGLLVAGLCSRVHCVPADDPASKIVTLKDQIKKLPAHNTAVVSSNTDFAFSLYKQLALTNPHENVIFSPLSVSMALAFLSLGARGPTLTELLEGLKFNLTKTPEAEIHQGFQHLLSTLDRSSNLLQLRLGNAMFIDEQLELLDKFVQDAHELYHSEAFPTNFQDLEAARRLINDYVKNKTEGKIVDLFKKLDPLTKVVLVNYIYFKAKWKTPFNPNLTTEADFHVSKNRTVRVPMMGIRALTVPYFRDEELACTVVELPYTSNDSALFILPDDGRMAAVEAKLLPETLRRWRDFLQPRWIVELYLPKFSISSDYRLHEILPQLGIEEIFGDNANLSGITNTKPLKVSQVVHSAVLDVNEEGTEAAAATGIDINVRSLERIALHFNRPFLFVIISKDIQSIIFLGKVTKP

SEQ ID NO: 24 - protein, 400 aa, Homo sapiens
HPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 25 - protein, 398 aa, Homo sapiens
NSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 26 - protein, 398 aa, Mus musculus
FPDGTLGMDAAVQEDHDNGTQLDSLTLASINTDFAFSLYKELVLKNPDKNIVFSPLSISAALAVMSLGAKGNTLEEILEGLKFNLTETSEADIHQGFGHLLQRLNQPKDQVQISTGSALFIEKRQQILTEFQEKARALYQAEAFTADFQQPRQAKKLINDYVRKQTQGMIKELVSDLDKRTLMVLVNYIYFKAKWKVPFDPLDTFKSEFYAGKRRPVIVPMMSMEDLTTPYFRDEELFCTVVELKYTGNASAMFILPDQGKMQQVEASLQPETLRKWKNSLKPRMIDELHLPKFSISTDYSLEDVLSKLGIREVFSTQADLSAITGTKDLRVSQVVHKAVLDVAETGTEAAAATGVKFVPMSAKLYPLTVYFNRPFLIMIFDTETEIAPFIAKIANPK

SEQ ID NO: 27 - protein, 418 aa, Mus musculus
MAFIAALGLLMAGICPAVLCFPDGTLGMDAAVQEDHDNGTQLDSLTLASINTDFAFSLYKELVLKNPDKNIVFSPLSISAALAVMSLGAKGNTLEEILEGLKFNLTETSEADIHQGFGHLLQRLNQPKDQVQISTGSALFIEKRQQILTEFQEKAKTLYQAEAFTADFQQPRQAKKLINDYVRKQTQGMIKELVSDLDKRTLMVLVNYIYFKAKWKVPFDPLDTFKSEFYAGKRRPVIVPMMSMEDLTTPYFRDEELSCTVVELKYTGNASALFILPDQGRMQQVEASLQPETLRKWKNSLKPRMIDELHLPKFSISTDYSLEDVLSKLGIREVFSTQADLSAITGTKDLRVSQVVHKAVLDVAETGTEAAAATGVKFVPMSAKLYPLTVYFNRPFLIMIFDTETEIAPFIAKIANPK

SEQ ID NO: 28 - protein, 398 aa, Mus musculus
FPDGTLGMDAAVQEDHDNGTQLDSLTLASINTDFAFSLYKELVLKNPDKNIVFSPLSISAALAVMSLGAKGNTLEEILEGLKFNLTETSEADIHQGFGHLLQRLNQPKDQVQISTGSALFIEKRQQILTEFQEKAKTLYQAEAFTADFQQPRQAKKLINDYVRKQTQGMIKELVSDLDKRTLMVLVNYIYFKAKWKVPFDPLDTFKSEFYAGKRRPVIVPMMSMEDLTTPYFRDEELSCTVVELKYTGNASALFILPDQGRMQQVEASLQPETLRKWKNSLKPRMIDELHLPKFSISTDYSLEDVLSKLGIREVFSTQADLSAITGTKDLRVSQVVHKAVLDVAETGTEAAAATGVKFVPMSAKLYPLTVYFNRPFLIMIFDTETEIAPFIAKIANPK

SEQ ID NO: 29 - DNA, 1272 bp, Homo sapiens
atggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctag

SEQ ID NO: 30 - DNA, 1257 bp, Mus musculus
atggccttcattgcagctctggggctcttgatggctgggatctgccctgctgtcctctgcttcccagatggcacgttgggaatggatgctgcagtccaagaagaccatgacaatgggacacaactggacagtctcacattggcctccatcaacactgactttgccttcagcctctacaaggagctggttttgaagaatccagataaaaatattgtcttctccccacttagcatctcagcggccttggccgtcatgtccctgggagcaaagggcaacaccctggaagagattctagaaggtctcaagttcaatcttacagagacctctgaggcagacatccaccagggctttgggcacctcctacagaggctcaaccagccaaaggaccaggtacagatcagcacgggtagtgccctgtttattgaaaagcgccagcagatcctgacagaattccaggagaaggcaagggctctgtaccaggctgaggccttcacagcagacttccagcagcctcgtcaggccaaaaagctcatcaatgactatgtgaggaaacagacccaggggatgatcaaggaactggtctcagacctggataaaaggacattgatggtgctggtgaattatatctactttaaagccaaatggaaggtgccctttgaccctcttgacacgttcaagtctgagttctacgcgggcaagaggaggcccgtgatagtgcccatgatgagcatggaggacctgaccacaccctacttccgagatgaggagcttttctgcactgtggtggagctgaagtacacaggaaatgccagtgccatgttcatcctccctgaccagggcaagatgcagcaggtggaagccagcttgcaaccagagaccctgaggaagtggaagaattctctgaaacccaggatgatagatgagctccacctgcccaagttctccatctccaccgactacagcctggaggatgtcctttcaaagctgggcatcagggaagtcttctccacacaggctgacctgtctgcaatcacaggaaccaaggatctgagagtctctcaggtggtccacaaggctgtgctggacgtggctgagacaggcacagaagcagctgctgccactggagtcaaatttgtcccaatgtctgcgaaactgtaccctctgactgtatatttcaatcggcctttcctgataatgatctttgacacagaaactgaaattgccccctttatagccaagatagccaaccccaaatga

SEQ ID NO: 31 - DNA, 1227 bp, Rattus norvegicus
atggatgggatcggctctgctctcctctccttcccagattgcatactgggagaggacactctattccatgaagaccaagacaaggggacacaactggacagtctcacattggcctccatcaatactgactttgccttcagcctctacaagaagctggctttgaggaatccacataaaaatgttgtcttctccccacttagcatctcagccgccttggccgtcgtgtccctgggagcaaagggcagcagcatggaagagattctagaaggtctcaagttcaatctcacagagacccctgagacagaaatccaccggggctttggacacctcctccagaggctcagccagccaagggacgagatacagatcagtacaggcaatgccctgtttattgaaaaacgccttcaggtcctggcagagttccaggagaaggcaaaggctctgtaccaagctgaggccttcacagctgatttccagcagtctcgtgaggccaaaaagctcatcaatgactatgtgagtaaacagacccaggggaagatccagggactgatcacaaacctagctaagaagacatccatggtactggtgaattacatctactttaaaggcaaatggaaggtgccttttgaccctcgggacacattccagtctgagttctactctggcaaaaggaggtctgtgaaagtgcccatgatgaagcttgaggacctgaccacaccctacgtccgggatgaggagctgaactgcactgttgtggagctgaagtacacaggaaatgccagcgccctgtttatcctccctgaccagggcaagatgcagcaggtggaagccagcttgcaaccagagaccctgaggagatggaaggactctctcaggcccagcatgatagatgagctctacctgcccaagttctccatctctgctgactacaacctggaggacgtccttccagagctgggcatcaaagaagtcttctccacacaggctgacctgtctgggatcacaggggataaggacctgatggtctttcaggtggtccacaaggctgttctggatgtggctgagacaggcacagaagcagccgctgccacaggggtcaaatttgttccaatgtctgcaaaactggaccctctgattatagctttcgaccggcctttcctgatgattatctctgacacagaaactgcaatagctccctttttggccaagatatttaaccccaaatga

SEQ ID NO: 32 - DNA, 20 bp, Artificial Sequence
  Primer
gctccagtgaattcggaaag

SEQ ID NO: 33 - DNA, 20 bp, Artificial Sequence
  Primer
gattatggctcagggtccaa

SEQ ID NO: 34 - DNA, 21 bp, Artificial Sequence
  Primer
tgagcaccttcttttccttca

SEQ ID NO: 35 - DNA, 22 bp, Artificial Sequence
  Primer
ttgtctaatgggaacgtcacac

SEQ ID NO: 36 - DNA, 26 bp, Artificial Sequence
  Primer
tctaattcatatcttcaaccaagagg

SEQ ID NO: 37 - DNA, 20 bp, Artificial Sequence
  Primer
tggtccttagccactccttc

SEQ ID NO: 38 - DNA, 20 bp, Artificial Sequence
  Primer
ctaaggccaaccgtgaaaag

SEQ ID NO: 39 - DNA, 20 bp, Artificial Sequence
  Primer
accagaggcatacagggaca